(19) United States
(12) Patent Application Publication
(10) Pub. No.: US 2008/0053430 A1
(43) Pub. Date: Mar. 6, 2008

(54) AEROSOL FORMULATION FOR THE INHALATION OF BETA AGONISTS

(76) Inventors: Michael Nowak, Walluf (DE); Michael Aven, Mainz (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/842,472

(22) Filed: Aug. 21, 2007

(30) Foreign Application Priority Data
Aug. 22, 2006 (EP) 06119274.6

(51) Int. Cl. A61M 11/00; A61K 31/535; A61K 31/56; A61P 11/00; A61K 31/58

(52) U.S. Cl. 128/200.14; 514/174; 514/178; 514/230.5

(57) ABSTRACT

The present invention relates to a propellant-free aerosol formulation which contains one or more compounds of general formula 1 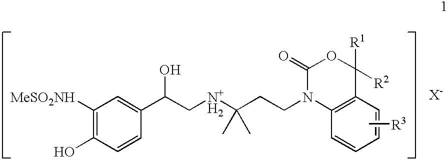
wherein the groups R¹, R², R³ and X⁻ may have the meanings indicated in the claims and in the specification, for inhalation.

This application claims priority benefit from EP 06 119 274.6, filed Aug. 22, 2006, which is incorporated herein in its entirety.

The present invention relates to a propellant-free aerosol formulation which contains one or more compounds of general formula 1 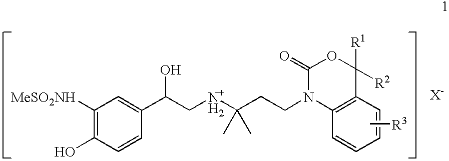
wherein the groups R¹, R², R³ and X⁻ may have the meanings indicated in the claims and in the specification, for inhalation.

### BACKGROUND TO THE INVENTION

Betamimetics (β-adrenergic substances) are known from the prior art. In this respect reference may be made for example to the disclosure of U.S. Pat. No. 4,341,778 or EP 43940 which proposes betamimetics for the treatment of a wide range of ailments.

For drug treatment of diseases it is often desirable to prepare medicaments with a longer duration of activity. As a rule, this ensures that the concentration of the active substance in the body needed to achieve the therapeutic effect is maintained for a longer period without the need to re-administer the drug at frequent intervals. Moreover, giving an active substance at longer time intervals contributes to the well-being of the patient to a high degree. It is particularly desirable to prepare a pharmaceutical composition which can be used therapeutically by administration once a day (single dose). The use of a drug once a day has the advantage that the patient can become accustomed relatively quickly to regularly taking the drug at certain times of the day.

The aim of the present invention is therefore to prepare medicament formulations for inhalation which on the one hand provide a therapeutic benefit in the treatment of respiratory complaints and are also characterised by a longer duration of activity and can thus be used to prepare medicaments with a longer duration of activity.

### DETAILED DESCRIPTION OF THE INVENTION

To solve the problem stated above the present invention proposes the following medicament formulations. The medicament formulations according to the invention are propellant-free medicament formulations, containing as sole active substance one or more compounds of general formula 1 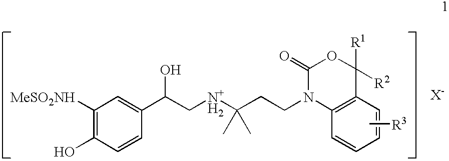
wherein

R¹ and R² which may be identical or different, denote hydrogen, halogen, C₁₋₄-alkyl or together denote C₁₋₆-alkylene and

R³ denotes hydrogen, halogen, OH, C₁₋₄-alkyl or O-C₁₋-alkyl;

X⁻ is one or more hydrogen cations and a mono- or polysubstituted negatively charged anion, preferably a mono- or polysubstituted negatively charged anion selected from among chloride, bromide, iodide, sulphate, phosphate, methanesulphonate, nitrate, maleate, acetate, benzoate, citrate, salicylate, trifluoroacetate, fumarate, tartrate, oxalate, succinate, benzoate and p-toluenesulphonate,

optionally in the form of the tautomers, enantiomers, mixtures of enantiomers, racemates or solvates thereof, at least one pharmacologically acceptable acid, optionally further pharmacologically acceptable excipients and/or complexing agents and water, ethanol or a mixture of water and ethanol as solvent.

Preferred medicament formulations contain compounds of general formula 1, wherein

R¹ and R² which may be identical or different, denote hydrogen, fluorine, chlorine, methyl, ethyl, propyl, butyl or together denote —CH₂—CH₂, —CH₂—CH₂—CH₂, —CH₂—CH₂—CH₂—CH₂ or —CH₂—CH₂—CH₂—CH₂—CH₂—;

R³ denotes hydrogen, fluorine, chlorine, OH, methyl, ethyl, methoxy, or ethoxy

X⁻ is a mono- or polysubstituted negatively charged anion, preferably a mono- or polysubstituted negatively charged anion selected from among chloride, bromide, iodide, sulphate, phosphate, methanesulphonate, nitrate, maleate, acetate, benzoate, citrate, salicylate, trifluoroacetate, fumarate, tartrate, oxalate, succinate, benzoate and p-toluenesulphonate,

optionally in the form of the tautomers, enantiomers, mixtures of enantiomers, racemates or solvates thereof.

Preferred medicament formulations contain compounds of general formula 1 contain, wherein

R¹ and R² which may be identical or different, denote hydrogen, methyl, ethyl, propyl or together denote —CH₂—CH₂, —CH₂—CH₂—CH₂, —CH₂—CH₂—CH₂—CH₂ or —CH₂—CH₂—CH₂—CH₂—CH₂—;

R³ denotes hydrogen, fluorine, OH, methyl or methoxy.

X⁻ is a mono- or polysubstituted negatively charged anion selected from among chloride, bromide, sulphate, methanesulphonate, maleate, acetate, benzoate, citrate, salicylate, trifluoroacetate, fumarate, tartrate and succinate;

optionally in the form of the tautomers, enantiomers, mixtures of enantiomers, racemates or solvates thereof.

Preferred medicament formulations contain compounds of general formula 1, wherein

R¹ and R² which may be identical or different, denote ethyl or propyl or together denote —CH₂—CH₂, —CH₂—CH₂—CH₂, —CH₂—CH₂—CH₂—CH₂ or —CH₂—CH₂—CH₂—CH₂—CH₂—;

R³ denotes hydrogen, fluorine, OH, methyl or methoxy,

optionally in the form of the tautomers, enantiomers, mixtures of enantiomers, racemates or solvates thereof.

Preferred medicament formulations contain compounds of general formula 1, wherein

R¹ and R² denote ethyl or propyl or together denote —CH₂—CH₂, —CH₂—CH₂—CH₂, —CH₂—CH₂—CH₂—CH₂ or —CH₂—CH₂—CH₂—CH₂—CH₂—;

R³ denotes hydrogen, fluorine, OH or methoxy,

optionally in the form of the tautomers, enantiomers, mixtures of enantiomers, racemates or solvates thereof.

Also preferred are medicament formulations which contain compounds of general formula 1 selected from among:
1.1: N-(5-{2-[1,1-dimethyl-3-(4-methyl-2-oxo-4H-benzo[d][1,3]oxazin-1-yl)-propylamino]-1-hydroxy-ethyl}-2-hydroxy-phenyl)-methanesulphonamide
1.2: N-(5-{2-[1,1-dimethyl-3-(2-oxo-4H-benzo[d][1,3]oxazin-1-yl)-propylamino]-1-hydroxy-ethyl}-2-hydroxy-phenyl)-methanesulphonamide
1.3: N-(5-{2-[3-(4-ethyl-2-oxo-4H-benzo[d][1,3]oxazin-1-yl)-1,1-dimethylpropylamino]-1-hydroxy-ethyl}-2-hydroxy-phenyl)-methanesulphonamide
1.4: N-(5-{2-[3-(4,4-dimethyl-2-oxo-4H-benzo[d][1,3]oxazin-1-yl)-1,1-dimethyl-propylamino]-1-hydroxy-ethyl}-2-hydroxy-phenyl)-methanesulphonamide
1.5: N-(2-hydroxy-5-{1-hydroxy-2-[3-(6-hydroxy-4,4-dimethyl-2-oxo-4H-benzo[d][1,3]oxazin-1-yl)-1,1-dimethyl-propylamino]-ethyl}-phenyl)-methanesulphonamide
1.6: N-(2-hydroxy-5-{1-hydroxy-2-[3-(6-methoxy-4,4-dimethyl-2-oxo-4H-benzo[d][1,3]oxazin-1-yl)-1,1-dimethyl-propylamino]-ethyl}-phenyl)-methanesulphonamide
1.7: N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide
1.8: N-[5-(2-{1,1-dimethyl-3-[spiro(cyclohexane-1,4′-2H-3′,1′-benzoxazin)-2′-oxo-1-yl]-propylamino}-1-hydroxy-ethyl)-2-hydroxy-phenyl]-methanesulphonamide
1.9: N-[5-(2-{1,1-dimethyl-3-[spiro(cyclopropyl-1,4′-2H-3′,1′-benzoxazin)-2′-oxo-1-yl]-propylamino}-1-hydroxy-ethyl)-2-hydroxy-phenyl]-methanesulphonamide
1.10: N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methanesulphonamide:
1.11: N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide
1.12: N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide
1.13: N-(5-{2-[3-(4,4-diethyl-8-methoxy-2-oxo-4H-benzo[d][1,3]oxazin-1-yl)-1,1-dimethyl-propylamino]-1-hydroxy-ethyl}-2-hydroxy-phenyl)-methanesulphonamide
1.14: N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methanesulphonamide
   in each case in the form of an acid addition salt with an acid HX, wherein X⁻ may have one of the meanings given above, and optionally in the form of the tautomers, enantiomers, mixtures of enantiomers, racemates or solvates thereof.

Also preferred are medicament formulations which contain compounds of general formula 1 which are selected from among:
1.7: N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide
1.8: N-[5-(2-{1,1-dimethyl-3-[spiro(cyclohexane-1,4′-2H-3′,1′-benzoxazin)-2′-oxo-1-yl]-propylamino}-1-hydroxy-ethyl)-2-hydroxy-phenyl]-methanesulphonamide
1.9: N-[5-(2-{1,1-dimethyl-3-[spiro(cyclopropyl-1,4′-2H-3′,1′-benzoxazin)-2′-oxo-1-yl]-propylamino}-1-hydroxy-ethyl)-2-hydroxy-phenyl]-methanesulphonamide
1.10: N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methanesulphonamide:
1.11: N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide
1.12: N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide
1.13: N-(5-{2-[3-(4,4-diethyl-8-methoxy-2-oxo-4H-benzo[d][1,3]oxazin-1-yl)-1,1-dimethyl-propylamino]-1-hydroxy-ethyl}-2-hydroxy-phenyl)-methanesulphonamide
1.14: N-(5-{2-[3-(4,4-diethyl-6-methoxy-2-oxo-4H-benzo[d][1,3]oxazin-1-yl)-1,1-dimethyl-propylamino]-1-hydroxy-ethyl}-2-hydroxy-phenyl)-methanesulphonamide
   in each case in the form of an acid addition salt with an acid HX, wherein X⁻ may have one of the meanings given above, and optionally in the form of the tautomers, enantiomers, mixtures of enantiomers, racemates or solvates thereof.

The medicament formulations according to the invention contain as solvent pure water, pure ethanol or mixtures of ethanol and water. If ethanol-water mixtures are used, the percentage by mass of ethanol in these mixtures is preferably in the range between 5 and 99% ethanol, particularly preferably in the range from 10 to 96% ethanol. Most particularly preferred medicament formulations for the purposes of the present invention contain as solvent pure water, pure ethanol or ethanol-water mixtures containing between 50 and 92%, particularly preferably between 69 and 91% ethanol.

If desired, other co-solvents may be used in addition to ethanol and water. Preferably, however, no other solvent is used according to the invention.

Within the scope of the present invention it is particularly preferable to use those compounds of formula 1 wherein X⁻ is selected from among chloride, maleate, salicylate, fumarate or succinate, optionally in the form of the hydrates and solvates thereof. Particularly preferred within the scope of the present invention are those formulations that contain the compound of formula 1, wherein X⁻ denotes chloride.

References to the compound of formula 1 always include within the scope of the present invention all possible amorphous and crystalline modifications of this compound. References to the compound of formula 1 also include within the scope of the present invention all the possible solvates and hydrates which may be formed from this compound.

Any reference to the compound 1′ within the scope of the present invention is to be regarded as a reference to the pharmacologically active free base of the following formula 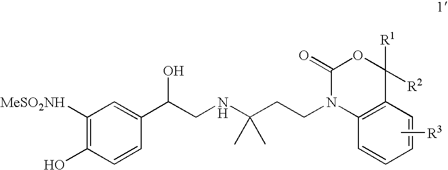
contained in the salts 1,
wherein the groups R¹, R² and R³ may have the meanings given above. In another aspect the present invention relates to medicament formulations containing as the sole active substance a free base of formula 1′ wherein the groups R¹, R² and R³ may have the meanings given above, optionally in the form of the tautomers, enantiomers, mixtures of enantiomers, racemates or solvates thereof, at least one pharmacologically acceptable acid, optionally further pharmacologically acceptable excipients and/or complexing agent and water, ethanol or a mixture of water and ethanol as solvent.

In another aspect the present invention relates to medicament formulations that contain the above-mentioned compounds of formula 1 in the form of the individual optical isomers, mixtures of the individual enantiomers or racemates. Particularly preferred are medicament formulations that contain the above-mentioned compounds of formula 1 in the form of the enantiomerically pure compounds, while the R-enantiomers of the compounds of formula 1 according to the invention are of exceptional importance. These R-enantiomers can also be represented by general formula R-1, 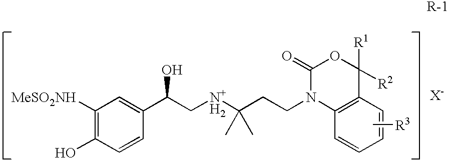
wherein the groups R¹, R², R³ and X⁻ may have the meanings given above.

Terms and Definitions Used

By the term “C₁₋₄-alkyl” (including those which are part of other groups) are meant branched and unbranched alkyl groups with 1 to 4 carbon atoms. Examples include: methyl, ethyl, n-propyl, iso-propyl, n-butyl, iso-butyl, sec-butyl or tert-butyl. The following abbreviations may optionally also be used for the above-mentioned groups: Me, Et, n-Pr, i-Pr, n-Bu, i-Bu, t-Bu, etc. Unless stated otherwise, the definitions propyl and butyl include all the possible isomeric forms of the groups in question. Thus, for example, propyl includes n-propyl and iso-propyl, butyl includes iso-butyl, sec-butyl and tert-butyl etc.

By the term “C₁₋₆-alkylene” (including those which are part of other groups) are meant branched and unbranched alkylene groups with 1 to 6 carbon atoms and by the term “C₁₋₄-alkylene” are meant branched and unbranched alkylene groups with 1 to 4 carbon atoms. Alkylene groups with 1 to 4 carbon atoms are preferred. Examples include: methylene, ethylene, propylene, 1-methylethylene, butylene, 1-methylpropylene, 1,1-dimethylethylene, 1,2-dimethylethylene, pentylene, 1,1-dimethylpropylene, 2,2-dimethylpropylene, 1,2-dimethylpropylene, 1,3-dimethylpropylene or hexylene. Unless stated otherwise, the definitions propylene, butylene, pentylene and hexylene include all the possible isomeric forms of the groups in question with the same number of carbons. Thus, for example, propyl also includes 1-methylethylene and butylene includes 1-methylpropylene, 1,1-dimethylethylene, 1,2-dimethylethylene.

“Halogen” within the scope of the present invention denotes fluorine, chlorine, bromine or iodine. Unless stated to the contrary, fluorine, chlorine and bromine are regarded as preferred halogens.

The term enantiomerically pure within the scope of the present invention describes compounds of formula 1 which are present in an enantiomeric purity of at least 85% ee, preferably at least 90% ee, particularly preferably >95% ee. The term ee (enantiomeric excess) is known in the art and describes the optical purity of chiral compounds.

Indications

In another aspect the present invention relates to the use of the medicament formulations according to the invention for preparing a pharmaceutical composition for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably the medicament formulations according to the invention are used as specified above for preparing a pharmaceutical composition for the treatment of obstructive pulmonary diseases selected from among bronchial asthma, paediatric asthma, severe asthma, acute asthma attacks, chronic bronchitis and chronic obstructive pulmonary disease (COPD), while it is particularly preferable according to the invention to use them for preparing a pharmaceutical composition for the treatment of bronchial asthma or COPD.

It is also preferable to use the medicament formulations according to the invention for preparing a pharmaceutical composition for the treatment of pulmonary emphysema which has its origins in COPD (chronic obstructive pulmonary disease) or α1-proteinase inhibitor deficiency.

It is also preferable to use the medicament formulations according to the invention for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

It is also preferable to use the medicament formulations according to the invention for preparing a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic scleroderma or sarcoidosis, granulomatoses, such as for example Boeck's disease, idiopathic interstitial pneumonia or idiopathic pulmonary fibrosis (IPF).

It is also preferable to use the medicament formulations according to the invention for preparing a pharmaceutical composition for the treatment of cystic fibrosis or mucoviscidosis.

It is also preferable to use the medicament formulations according to the invention for preparing a pharmaceutical composition for the treatment of bronchitis, such as for example bronchitis caused by bacterial or viral infection, allergic bronchitis and toxic bronchitis.

It is also preferable to use the medicament formulations according to the invention for preparing a pharmaceutical composition for the treatment of bronchiectasis.

It is also preferable to use the medicament formulations according to the invention for preparing a pharmaceutical composition for the treatment of ARDS (adult respiratory distress syndrome).

It is also preferable to use the medicament formulations according to the invention for preparing a pharmaceutical composition for the treatment of pulmonary oedema, for example toxic pulmonary oedema after aspiration or inhalation of toxic substances and foreign substances.

Most preferably, the present invention relates to the use of the pharmaceutical formulations according to the invention for preparing a pharmaceutical composition for the treatment of asthma or COPD. Also of particular importance is the above-mentioned use for preparing a pharmaceutical composition for once-a-day treatment of inflammatory and obstructive respiratory complaints, particularly for the once-a-day treatment of asthma or COPD.

Moreover the present invention relates to a process for the treatment of the above-mentioned diseases, characterised in that one or more of the above-mentioned medicament formulations according to the invention are administered in therapeutically effective amounts.

Formulation

The present invention relates to liquid active substance formulations of these compounds which can be administered by inhalation; the liquid formulations according to the invention have to meet high quality standards. The formulations according to the invention may be inhaled by oral or nasal route. To achieve an optimum distribution of the active substances in the lung it makes sense to use a liquid formulation without propellant gases administered using suitable inhalers. A formulation of this kind may be inhaled both by oral route and by nasal route. Those inhalers which are capable of nebulising a small amount of a liquid formulation in the dosage needed for therapeutic purposes within a few seconds into an aerosol suitable for therapeutic inhalation are particularly suitable. Within the scope of the invention, preferred nebulisers are those in which an amount of less than 100 microlitres, preferably less than 50 microlitres, most preferably less than 25 microlitres of active substance solution can be nebulised preferably in one puff or two puffs to form an aerosol having an average particle size (or particle diameter) of less than 20 microns, preferably less than 10 microns, so that the inhalable part of the aerosol already corresponds to the therapeutically effective quantity.

An apparatus of this kind for the propellant-free administration of a metered amount of a liquid pharmaceutical composition for inhalation is described in detail for example in International Patent Application WO 91/14468 “Atomizing Device and Methods” and also in WO 97/12687, cf. FIGS. 6a and 6b and the accompanying description. In a nebuliser of this kind a pharmaceutical solution is converted by means of a high pressure of up to 500 bar into an aerosol destined for the lungs, which is sprayed. Within the scope of the present specification reference is expressly made to the entire contents of the literature mentioned above. In inhalers of this kind the formulations of solutions are stored in a reservoir. It is essential that the active substance formulations used are sufficiently stable when stored and at the same time are such that they can be administered directly, if possible without any further handling, in accordance with their medical purpose. Moreover, they must not contain any ingredients which might interact with the inhaler in such a way as to damage the inhaler or the pharmaceutical quality of the solution or of the aerosol produced.

To nebulise the solution a special nozzle is used as described for example in WO 94/07607 or WO 99/16530. Reference is expressly made here to both these publications.

The aim of the present invention is to provide an aqueous, ethanolic or aqueous-ethanolic formulation of the compound of formula 1 which meets the high standards required to ensure optimum nebulisation of a solution using the inhalers mentioned above. The active substance formulations according to the invention must be of sufficiently high pharmaceutical quality, i.e. they should be pharmaceutically stable over a storage time of some years, preferably at least one year, more preferably two years.

These propellant-free formulations of solutions must also be capable of being nebulised by means of an inhaler under pressure, while the composition delivered in the aerosol produced is reproducibly within a specified range.

According to the invention the formulation preferably contains only one compound of formula 1. However, the formulation may also contain a mixture of different salts of formula 1. If the medicament formulations according to the invention contain different salts of formula 1 the preferred formulations according to the invention are those wherein the various salts constitute different salts of the same free base of formula 1′.

Formulations which contain active substances other than those of formula 1 are not covered by the invention.

The concentration of the compound of formula 1 based on the amount of pharmacologically active free base 1′ in the medicament formulation according to the invention is about 0.1 to 1600 mg per 100 ml, preferably about 0.5 to 1000 mg per 100 ml, particularly preferably 0.75 to 200 mg per 100 ml according to the invention. Particularly preferably 100 ml of the formulations according to the invention contain about 1 to about 100 mg of 1′.

The pH of the formulation according to the invention is preferably in a range from 2.0 to 6.5, preferably between 2.2 and 5.0, particularly preferably between about 3.0 and 4.5.

The pH is adjusted by the addition of pharmacologically acceptable acids. Pharmacologically acceptable inorganic acids or organic acids may be used for this purpose. Examples of preferred inorganic acids are selected from the group consisting of hydrochloric acid, hydrobromic acid, nitric acid, sulphuric acid and phosphoric acid.

Examples of particularly suitable organic acids are selected from the group consisting of ascorbic acid, citric acid, malic acid, tartaric acid, maleic acid, succinic acid, fumaric acid, acetic acid, formic acid, propionic acid, methanesulphonic acid and benzenesulphonic acid. Preferred inorganic acids are hydrochloric acid and sulphuric acid, of which hydrochloric acid is particularly preferred according to the invention. Of the organic acids, ascorbic acid, fumaric acid and citric acid are preferred, while citric acid is particularly preferred according to the invention. If desired, mixtures of the abovementioned acids may also be used, particularly in the case of acids which have other properties in addition to their acidifying properties, e.g. those which act as flavourings or antioxidants, such as for example citric acid or ascorbic acid. If desired, pharmacologically acceptable bases may also be used to titrate the pH precisely. Suitable bases include for example alkali metal hydroxides and alkali metal carbonates. The preferred alkali metal ion is sodium. If bases of this kind are used, care must be taken to ensure that the resulting salts, which are then contained in the finished pharmaceutical formulation, are pharmacologically compatible with the abovementioned acid.

The formulations according to the invention may contain complexing agents as further constituents. By complexing agents are meant within the scope of the present invention molecules which are capable of entering into complex bonds. Preferably, these compounds should have the effect of complexing cations, most preferably metal cations. The formulations according to the invention preferably contain editic acid (EDTA) or one of the known salts thereof, e.g. sodium EDTA or disodium EDTA, as complexing agent. Preferably, disodium edetate is used, optionally in the form of its hydrates, more preferably in the form of its dihydrate. If complexing agents are used within the formulations according to the invention, their content is preferably in the range from 1 to 50 mg per 100 ml, more preferably in the range from 2 to 15 mg per 100 ml of the formulation according to the invention. Preferably, the formulations according to the invention contain a complexing agent in an amount of about 4 to 12 mg per 100 ml, particularly preferably about 10 mg per 100 ml of the formulation according to the invention.

The remarks made concerning disodium edetate also apply analogously to other possible additives which are comparable to EDTA or the salts thereof, which have complexing properties and can be used instead of them, such as for example nitrilotriacetic acid and the salts thereof.

Other pharmacologically acceptable excipients may also be added to the formulation according to the invention. By adjuvants and additives are meant, in this context, any pharmacologically acceptable and therapeutically useful substance which is not an active substance, but can be formulated together with the active substance in the pharmacologically suitable solvent, in order to improve the qualities of the active substance formulation. Preferably, these substances have no pharmacological effects or no appreciable or at least no undesirable pharmacological effects in the context of the desired therapy. The adjuvants and additives include, for example, stabilisers, antioxidants and/or preservatives which prolong the shelf life of the finished pharmaceutical formulation, as well as flavourings, vitamins and/or other additives known in the art. The additives also include pharmacologically acceptable salts such as sodium chloride, for example. The preferred excipients include antioxidants such as ascorbic acid, for example, provided that it has not already been used to adjust the pH, propylgallate, BHA (butylhydroxyanisol), BHT (butylhydroxytoluene), TBHQ (tert.butylhydroxyquinone), vitamin A, vitamin E, α-tocopherol and similar vitamins or provitamins occurring in the human body, the preferred antioxidants being BHT and a!-tocopherol.

Preservatives can be used to protect the formulation from contamination with pathogenic bacteria. Suitable preservatives are those known from the prior art, particularly benzalkonium chloride or benzoic acid or benzoates such as sodium benzoate in the concentrations known from the prior art. Preferably, benzalkonium chloride is added to the formulation according to the invention. The amount of benzalkonium chloride is between 1 mg and 50 mg per 100 ml of formulation, preferably about 2 to 15 mg per 100 ml, more preferably about 3 to 12 mg per 100 ml, most preferably about 4 to 10 mg per 100 ml of the formulation according to the invention. Benzalkonium chloride may also be used according to the invention in admixture with other preservatives.

Preferred formulations contain only benzalkonium chloride, sodium edetate and the acid needed to adjust the pH, in addition to the solvent water and the compounds of formula 1.

Nebulisers

The nebulisation of pharmaceuticals dissolved or suspended in water may be carried out using compressed air or ultrasound. The resulting particle spectrum is superior to propellant gas and powder aerosols in its delivery to the lungs. This method of inhalation is suitable for cases of severe asthma and because of the simple inhalation technique it is also suitable for children and patients who have problems coordinating their breathing. There are both stationary devices and small devices for use when travelling. These are naturally always larger than MDI's and DPI's. The pharmaceutical preparations that can be used are limited to microbiologically safe, aqueous, isotonic and pH-neutral solutions or suspension.

Jet nebulisers—For a long time, simple devices have been used for distributing solutions, in which a powerful air current is passed through the opening of a capillary tube through which the solution is sucked (the perfume atomiser principle). In hand-held atomisers made of glass (nebulisers) the air current is generated by compressing a rubber ball or by pumping (pump atomiser). More recent stationary devices for aerosol therapy are nebulisers operating by compressed air which are able to generate an amount of over 50% in the optimum size range (1-5 μm). Compressed air is accelerated through a nozzle and carries the medicament solution through capillaries (Bernoulli effect), during which time the solution is dispersed. An impact plate located behind the nozzle additionally serves to break up the solution. Special blocking means ensure that only the smallest particles escape, while the larger particles flow back into the reservoir and can be nebulised again. During inhalation considerable evaporation takes place, which leads to a cool aerosol and concentration of the active substance solution, as a result of the coldness of evaporation.

Ultrasound nebulisers—A piezoelectric crystal is excited, by high-frequency alternating current, to produce vibrations which are transmitted through a transfer medium to the active substance solution and from it release very fine droplets of liquid but at the same time heat the liquid.

The medicament formulations according to the invention comprising compounds of formula 1 are preferably used in an inhaler of the type described hereinbefore to produce the propellant-free aerosols according to the invention. At this point we should once again expressly mention the patent documents described hereinbefore, to which reference is hereby made. As described at the beginning, a further developed embodiment of the preferred inhaler is disclosed in WO 97/12687 (cf. in particular FIGS. 6a and 6b and the associated passages of description). This nebuliser (Respimat®) can advantageously be used to produce the inhalable aerosols according to the invention. Because of its cylindrical shape and handy size of less than 9 to 15 cm long and 2 to 4 cm wide, the device can be carried anywhere by the patient. The nebuliser sprays a defined volume of the pharmaceutical formulation out through small nozzles at high pressures, so as to produce inhalable aerosols.

The preferred atomiser essentially consists of an upper housing part, a pump housing, a nozzle, a locking clamp, a spring housing, a spring and a storage container, characterised by
a pump housing fixed in the upper housing part and carrying at one end a nozzle body with the nozzle or nozzle arrangement,
a hollow piston with valve body,
a power take-off flange in which the hollow body is fixed and which is located in the upper housing part,
a locking clamping mechanism located in the upper housing part,
a spring housing with the spring located therein, which is rotatably mounted on the upper housing part by means of a rotary bearing,
a lower housing part which is fitted onto the spring housing in the axial direction.

The hollow piston with valve body corresponds to a device disclosed in WO 97/12687. It projects partially into the cylinder of the pump housing and is disposed to be axially movable in the cylinder. Reference is made particularly to FIGS. 1-4—especially FIG. 3—and the associated parts of the description of the above-mentioned International Patent Application. At the moment of release of the spring the hollow piston with valve body exerts, at its high pressure end, a pressure of 5 to 60 Mpa (about 50 to 600 bar), preferably 10 to 60 Mpa (about 100 to 600 bar) on the fluid, the measured amount of active substance solution. Volumes of 10 to 50 microlitres are preferred, volumes of 10 to 20 microlitres are more preferable, whilst a volume of 10 to 15 microlitres per actuation is particularly preferred.

The valve body is preferably mounted at the end of the hollow piston which faces the nozzle body. The nozzle in the nozzle body is preferably microstructured, i.e. manufactured by micro-engineering. Microstructured nozzle bodies are disclosed for example in WO 99/16530; reference is hereby made to the contents thereof, especially FIG. 1 and the associated description. The nozzle body consists for example of two sheets of glass and/or silicon securely fixed together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns and the length being 7 to 9 microns.

If there is a plurality of nozzle openings, preferably two, the directions of spraying of the nozzles in the nozzle body may run parallel to each other or may be inclined relative to one another in the direction of the nozzle opening. In the case of a nozzle body having at least two nozzle openings at the outlet end, the directions of spraying may be inclined relative to one another at an angle of 20 degrees to 160 degrees, preferably at an angle of 60 to 150 degrees, most preferably 80 to 100°. The nozzle openings are preferably arranged at a spacing of 10 to 200 microns, more preferably at a spacing of 10 to 100 microns, still more preferably 30 to 70 microns. A spacing of 50 microns is most preferred. The directions of spraying therefore meet in the region of the nozzle openings.

As already mentioned, the liquid pharmaceutical preparation hits the nozzle body at an entry pressure of up to 600 bar, preferably 200 to 300 bar and is atomised through the nozzle openings into an inhalable aerosol. The preferred particle sizes of the aerosol are up to 20 microns, preferably 3 to 10 microns.

The locking clamping mechanism contains a spring, preferably a cylindrical helical compression spring as a store for the mechanical energy. The spring acts on the power take-off flange as a spring member the movement of which is determined by the position of a locking member. The travel of the power take-off flange is precisely limited by an upper stop and a lower stop. The spring is preferably tensioned via a stepping-up gear, e.g. a helical sliding gear, by an external torque which is generated when the upper housing part is turned relative to the spring housing in the lower housing part. In this case, the upper housing part and the power take-off flange contain a single- or multi-speed spline gear.

The locking member with the engaging locking surfaces is arranged in an annular configuration around the power take-off flange. It consists for example of a ring of plastics or metal which is inherently radially elastically deformable. The ring is arranged in a plane perpendicular to the axis of the atomiser. After the tensioning of the spring, the locking surfaces of the locking member slide into the path of the power take-off flange and prevent the spring from being released. The locking member is actuated by means of a button. The actuating button is connected or coupled to the locking member. In order to actuate the locking clamping mechanism the actuating button is moved parallel to the annular plane, preferably into the atomiser, and the deformable ring is thereby deformed in the annular plane. Details of the construction of the locking clamping mechanism are described in WO 97/20590.

The lower housing part is pushed axially over the spring housing and covers the bearing, the drive for the spindle and the storage container for the fluid.

When the atomiser is operated, the upper part of the housing is rotated relative to the lower part, the lower part taking the spring housing with it. The spring meanwhile is compressed and biased by means of the helical sliding gear, and the clamping mechanism engages automatically. The angle of rotation is preferably a whole-number fraction of 360 degrees, e.g. 180 degrees. At the same time as the spring is tensioned, the power take-off component in the upper housing part is moved along by a given amount, the hollow piston is pulled back inside the cylinder in the pump housing, as a result of which some of the fluid from the storage container is sucked into the high pressure chamber in front of the nozzle. If desired, a plurality of replaceable storage containers containing the fluid to be atomised can be inserted in the atomiser one after another and then used. The storage container contains the aqueous aerosol preparation according to the invention.

The atomising process is initiated by gently pressing the actuating button. The clamping mechanism then opens the way for the power take-off component. The biased spring pushes the piston into the cylinder in the pump housing. The fluid emerges from the nozzle of the atomiser in the form of a spray.

Further details of the construction are disclosed in PCT applications WO 97/12683 and WO 97/20590, to which reference is hereby made.

The components of the atomiser (nebuliser) are made of a material suitable for their function. The housing of the atomiser and—if the function allows—other parts as well are preferably made of plastics, e.g. by injection moulding. For medical applications, physiologically acceptable materials are used.

FIGS. 6 a/b of WO 97/12687 show the nebuliser (Respimat®) with which the aqueous aerosol preparations according to the invention can advantageously be inhaled. FIG. 6 a shows a longitudinal section through the atomiser with the spring under tension, FIG. 6 b shows a longitudinal section through the atomiser with the spring released.

The upper housing part (51) contains the pump housing (52), on the end of which is mounted the holder (53) for the atomiser nozzle. In the holder is the nozzle body (54) and a filter (55). The hollow piston (57) fixed in the power take-off flange (56) of the locking clamping mechanism projects partly into the cylinder of the pump housing. At its end the hollow piston carries the valve body (58). The hollow piston is sealed off by the gasket (59). Inside the upper housing part is the stop (60) on which the power take-off flange rests when the spring is relaxed. Located on the power take-off flange is the stop (61) on which the power take-off flange rests when the spring is under tension. After the tensioning of the spring, the locking member (62) slides between the stop (61) and a support (63) in the upper housing part. The actuating button (64) is connected to the locking member. The upper housing part ends in the mouthpiece (65) and is closed off by the removable protective cap (66).

The spring housing (67) with compression spring (68) is rotatably mounted on the upper housing part by means of the snap-fit lugs (69) and rotary bearings. The lower housing part (70) is pushed over the spring housing. Inside the spring housing is the replaceable storage container (71) for the fluid (72) which is to be atomised. The storage container is closed off by the stopper (73), through which the hollow piston projects into the storage container and dips its end into the fluid (supply of active substance solution).

The spindle (74) for the mechanical counter is mounted on the outside of the spring housing. The drive pinion (75) is located at the end of the spindle facing the upper housing part. On the spindle is the slider (76).

The nebuliser described above is suitable for nebulising the aerosol preparations according to the invention to form an aerosol suitable for inhalation.

If the formulation according to the invention is nebulised using the method described above (Respimat ), the mass expelled, in at least 97%, preferably at least 98% of all the actuations of the inhaler (puff or puffs), should correspond to a defined quantity with a range of tolerance of not more than 25%, preferably 20% of this quantity. Preferably, between 5 and 30 mg, more preferably between 5 and 20 mg of formulation are delivered as a defined mass per puff.

The formulation according to the invention can also be nebulised using inhalers other than those described above, for example jet-stream inhalers.

The present invention also relates to an inhalation kit consisting of one of the pharmaceutical preparations according to the invention described above and an inhaler suitable for nebulising this pharmaceutical preparation. The present invention preferably relates to an inhalation kit consisting of one of the pharmaceutical preparations according to the invention described above and the Respimat® inhaler described above.

The examples of formulations given below serve as illustrations without restricting the subject matter of the present invention to the compositions shown by way of example.

### EXAMPLES

### Example 1

### N-(5-{2-[1,1-Dimethyl-3-(4-Methyl-2-Oxo-4H-Benzo[D][1,3]Oxazin-1-Yl)-Propylamino]-1-Hydroxy-Ethyl}-2-Hydroxy-Phenyl)-Methanesulphonamide

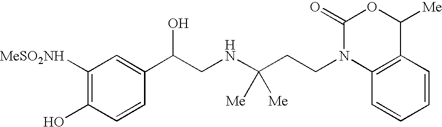

The compound is known from EP 43940. The individual diastereomers of this embodiment may be obtained by common methods known in the art.

### Example 2

### N-(5-{2-[1,1-Dimethyl-3-(2-Oxo-4H-Benzo[D][1,3]Oxazin-1-Yl)-Propylamino]-1-Hydroxy-Ethyl}-2-Hydroxy-Phenyl)-Methanesulphonamide

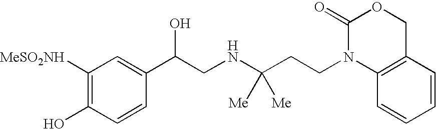

The compound is known from EP 43940. The (R)- and (S)-enantiomers of this embodiment may be obtained by common methods known in the art.

### Example 3

### N-(5-{2-[3-(4-Ethyl-2-Oxo-4H-Benzo[D][1,3]Oxazin-1-Yl)-1,1-Dimethyl-Propylamino]-1-Hydroxy-Ethyl}-2-Hydroxy-Phenyl)-Methanesulphonamide

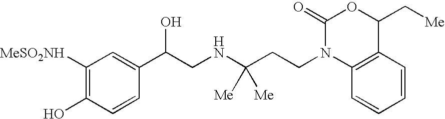

The compound is known from EP 43940. The individual diastereomers of this embodiment may be obtained by common methods known in the art.

### Example 4

### N-(5-{2-[3-(4,4-Dimethyl-2-Oxo-4H-Benzo[D][1,3]Oxazin-1-Yl)-1,1-Dimethyl-Propylamino]-1-Hydroxy-Ethyl}-2-Hydroxy-Phenyl)-Methanesulphonamide

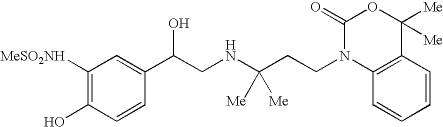

The compound is known from EP 43940. The (R)- and (S)-enantiomers of this embodiment may be obtained by common methods known in the art.

### Example 5

### N-(2-Hydroxy-5-{1-Hydroxy-2-[3-(6-Hydroxy-4,4-Dimethyl-2-Oxo-4H-Benzo[D][1,3]Oxazin-1-YL)-1,1-Dimethyl-Propylamino]-Ethyl}-Phenyl)-Methanesulphonamide

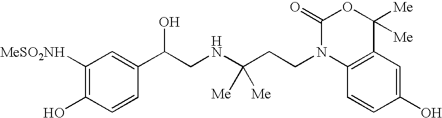

The compound is known from EP 43940. The (R)- and (S)-enantiomers of this embodiment may be obtained by common methods known in the art.

### Example 6

### N-(2-Hydroxy-5-{1-Hydroxy-2-[3-(6-Methoxy-4,4-Dimethyl-2-Oxo-4H-Benzo[D][1,3]Oxazin-1-YL)-1,1-Dimethyl-Propylamino]-Ethyl}-Phenyl)-Methanesulphonamide

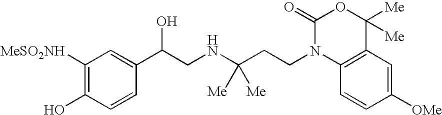

The compound is known from EP 43940. The (R)- and (S)-enantiomers of this embodiment may be obtained by common methods known in the art.

The examples of synthesis described below serve to illustrate new compounds according to the invention in more detail. However, they are intended only as examples of procedures to illustrate the invention without restricting it to the subject matter described in an exemplifying capacity hereinafter.

HPLC method (method A): Symmetry C18 (Waters): 3.5 pm; 4.6×150 mm; column temperature: 20° C.; gradient: acetonitrile/phosphate buffer (pH 7) 20:80→80:20 in 30 minutes; flow: 1.0 mL/min; detection at 220 and 254 nm.

### SYNTHESIS OF INTERMEDIATE PRODUCTS 1-7

### Intermediate Product 1

### 1-(3-amino-3-methyl-butyl)-4,4-dipropyl-1,4-dihydro-benzo[d][1,3]oxazin-2-one

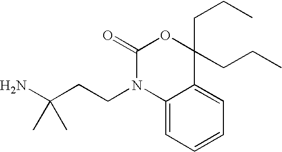

a) 4-(2-amino-phenyl)-heptan-4-ol: 90 mL (180.0 mmol) propylmagnesium chloride (2 M in ether) are added dropwise to a solution of 7.00 mL (54.0 mmol) methyl anthranilate in abs. THF (70 mL) at 020 C. within 30 minutes. The mixture is stirred for one hour at ambient temperature and then combined with 100 mL of 3 molar aqueous ammonium chloride solution and ethyl acetate. The phases are separated and the aqueous phase is exhaustively extracted with ethyl acetate. The combined organic phases are washed with potassium hydrogen carbonate solution and saturated sodium chloride solution and dried on sodium sulphate. The crude product is used in the next reaction step without further purification. Yield: 6.70 g (60%).

b) tert-butyl {3-[2-(1-hydroxy-1-propyl-butyl)-phenylamino]-1,1-dimethyl-propyl}-carbamate: 1.40 g (22.27 mmol) sodium cyanoborohydride are added to a solution of 3.10 g (14.05 mmol) 4-(2-amino-phenyl)-heptan-4-ol and 3.60 g (17.88 mmol) tert-butyl (1,1-dimethyl-3-oxo-propyl)-carbamate in methanol (40 mL) and acetic acid (6 mL). The mixture is stirred for 16 hours at ambient temperature, diluted with ethyl acetate, washed with 0.5 molar potassium hydrogen sulphate solution and saturated sodium chloride solution, dried on sodium sulphate and evaporated down in vacuo. The crude product is used in the next reaction step without further purification. Yield: 6.00 g (quantitative yield).

c) tert-butyl [1,1-dimethyl-3-(2-oxo-4,4-dipropyl-4H-benzo[d][1,3]oxazin-1-yl)-propyl]-carbamate: 8.85 mL (16.81 mmol) phosgene solution (20 wt. % in toluene) are slowly added dropwise at 0° C. to a solution of 6.00 g (15.28 mmol) tert-butyl {3-[2-(1-hydroxy-1-propyl-butyl)-phenylamino]-1,1-dimethyl-propyl}-carbamate and 5.32 mL (38.21 mmol) triethylamine in abs. THF (80 mL). The mixture is stirred for 2 hours at ambient temperature, diluted with ethyl acetate, combined with ice and made basic with saturated aqueous ammonia solution. The aqueous phase is exhaustively extracted with ethyl acetate and the combined organic phases are washed with saturated sodium chloride solution, dried on sodium sulphate and evaporated down in vacuo. After column chromatography (silica gel, cyclohexane/ethyl acetate=6:1) the product is obtained. Yield: 4.57 g (71%).

d) 1-(3-amino-3-methyl-butyl)-4,4-dipropyl-1,4-dihydro-benzo[d][1,3]oxazin-2-one: A solution of 4.20 g (10.03 mmol) tert-butyl [1,1-dimethyl-3-(2-oxo-4,4-dipropyl-4H-benzo[d][1,3]oxazin-1-yl)-propyl]-carbamate in 35 mL formic acid is stirred for 24 hours at ambient temperature and then poured onto ice. The aqueous phase is made basic with saturated aqueous ammonia solution and exhaustively extracted with ethyl acetate. The combined organic extracts are washed with sodium chloride solution, dried on sodium sulphate and evaporated down in vacuo. The residue is taken up in ethyl acetate (50 mL) and combined with 4 mL hydrochloric acid in ethyl acetate (saturated). The solution is evaporated down and twice mixed with a little ethanol and evaporated down in vacuo. Trituration of the residue with diisopropylether yields the product as the hydrochloride salt. Yield: 2.60 g (73%).

### Intermediate Product 2

### 1-(3-amino-3-methyl-butyl)-4,4-diethyl-7-fluoro-1,4-dihydro-benzo[d][1,3]oxazin-2-one

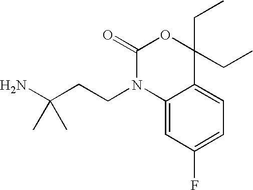

a) 3-(2-amino-4-fluoro-phenyl)-pentan-3-ol: The product is obtained analogously to intermediate product 1a by reacting methyl 2-amino-4-fluoro-benzoate and ethylmagnesium bromide in dichloromethane at −78° C. with heating to ambient temperature. Yield: 4.1 g (99%).

b) tert-butyl {3-[2-(1-ethyl-1-hydroxy-propyl)-5-fluoro-phenylamino]-1,1-dimethyl-propyl}-carbamate: The product is obtained analogously to intermediate product 1b starting from 3-(2-amino-4-fluoro-phenyl)-pentan-3-ol and tert-butyl (1,1-dimethyl-3-oxo-propyl)-carbamate. The crude product is purified by column chromatography (silica gel, dichloromethane/methanol=100:0→98:2). Yield: 7.70 g (99%).

c) tert-butyl [3-(4,4-diethyl-7-fluoro-2-oxo-4H-benzo[d][1,3]oxazin-1-yl)-1,1-dimethyl-propyl]-carbamate: The product is obtained analogously to intermediate product 1c starting from tert-butyl {3-[2-(1-ethyl-1-hydroxy-propyl)-5-fluoro-phenylamino]-1,1-dimethyl-propyl}-carbamate. Yield: 4.20 g (51%).

d) 1-(3-amino-3-methyl-butyl)-4,4-diethyl-7-fluoro-1,4-dihydro-benzo[d][1,3]oxazin-2-one: The product is prepared analogously to intermediate product 1d starting from tert-butyl [3-(4,4-diethyl-7-fluoro-2-oxo-4H-benzo[d][1,3]oxazin-1-yl)-1,1-dimethyl-propyl]-carbamate as the free base. Yield: 2.90 g (96%); ESI-MS: [M+H]⁺=309.

### Intermediate Product 3

### 1-(3-amino-3-methyl-butyl)-spiro(cyclopropyl-1,4′-2H-3′,1′-benzoxazin)-2′-one

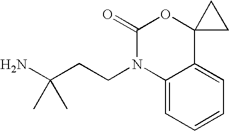

a) 1-(2-dibenzylamino-phenyl)-cyclopropanol: 2.45 mL (8.4 mmol) titanium tetraisopropoxide are slowly added dropwise at ambient temperature to a solution of 18.5 g (55.8 mmol) methyl 2-dibenzylamino-benzoate in 150 mL THF. After one hour's stirring 40.9 mL (122.7 mmol) ethylmagnesium bromide (3 M in diethyl ether) are added. The mixture is stirred for one hour, another 4 mL of 3 molar ethylmagnesium bromide solution are added and the mixture is stirred for 2 hours. The reaction mixture is combined with saturated ammonium chloride solution and extracted with ethyl acetate. The aqueous phase is combined with 1 molar hydrochloric acid until a clear solution is obtained and extracted with ethyl acetate. The combined organic phases are washed with sodium hydrogen carbonate solution and sodium chloride solution, dried on sodium sulphate and evaporated down. The residue is purified by chromatography (hexane/ethyl acetate=20:1). Yield: 10.0 g (54%).

b) 1-(2-amino-phenyl)-cyclopropanol: 9.90 g (30.1 mmol) 1-(2-dibenzylamino-phenyl)-cyclopropanol are dissolved in 70 mL methanol and hydrogenated in the presence of 1 g palladium on charcoal (10%) at 3 bar hydrogen pressure. The catalyst is removed by suction filtering, the filtrate is evaporated down and the residue is purified by chromatography (silica gel; cyclohexane/ethyl acetate=5:1). Yield: 1.80 g (40%).

c) tert-butyl {3-[2-(1-hydroxy-cyclopropyl)-phenylamino]-1,1-dimethyl-propyl}-carbamate: Prepared analogously to the method described for intermediate product 1b from 1.77 g (11.86 mmol) 1-(2-amino-phenyl)-cyclopropanol and 3.15 g (15.66 mmol) tert-butyl (1,1-dimethyl-3-oxo-propyl)-carbamate. The crude product obtained is purified by column chromatography (silica gel, cyclohexane/ethyl acetate 4:1). Yield: 2.60 g.

d) tert-butyl {1,1-dimethyl-3-[spiro(cycloproyl-1,4′-2H-3′,1′-benzoxazin)-2′-oxo-1-yl]-propyl}-carbamate: The product is obtained analogously to intermediate product 1c starting from 2.60 g (7.74 mmol) tert-butyl {3-[2-(1-hydroxy-cyclopropyl)-phenylamino]-1,1-dimethyl-propyl}-carbamate. A difference here is that there is no purification by column chromatography. Yield: 2.60 g.

e) 1-(3-amino-3-methyl-butyl)-spiro(cyclopropyl-1,4′-2H-3′,1′-benzoxazin)-2′-one: Obtained analogously to the method described for Intermediate 1d by reacting 3.10 g (8.60 mmol) tert-butyl {1,1-dimethyl-3-[spiro(cycloproyl-1,4′-2H-3′,1′-benzoxazin)-2′-oxo-1-yl]-propyl}-carbamate and 30 mL formic acid. Yield: 2.10 g (94%).

### Intermediate Product 4

### 1-(3-amino-3-methyl-butyl)-4,4-diethyl-1,4-dihydro-benzo[d][1,3]oxazin-2-one

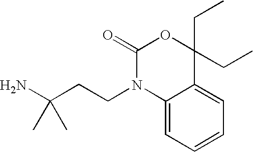

a) 3-(2-amino-phenyl)-pentan-3-ol: 100 mL of a 3 molar ethylmagnesium bromide solution in diethyl ether are added dropwise at −40° C. to a solution of 7.77 mL (60 mmol) 2-amino-methylbenzoic acid in 130 mL THF. The mixture is stirred overnight with heating to ambient temperature, combined with saturated ammonium chloride solution, acidified with 1 molar hydrochloric acid and extracted with ethyl acetate. The combined organic phases are extracted with water, dried on sodium sulphate and evaporated down. The crude product is further reacted directly. Yield: 10.9 g; mass spectroscopy: [M+H]⁺=180.

b) tert-butyl {3-[2-(1-ethyl-1-hydroxy-propyl)-phenylamino]-1,1-dimethyl-propyl}-carbamate: 3.16 g (47.7 mmol) sodium cyanoborohydride are added at ambient temperature to 5.70 g (31.8 mmol) 3-(2-amino-phenyl)-pentan-3-ol and 2.63 mL (47.7 mmol) acetic acid in 18 mL methanol. Then a solution of 7.04 g (35 mmol) tert-butyl (1,1-dimethyl-3-oxo-propyl)-carbamate in 18 mL methanol is slowly added dropwise. After the addition has ended the mixture is stirred for four hours, combined with 1 molar hydrochloric acid (development of gas) and then made basic with aqueous ammonia solution. It is extracted with ethyl acetate and the combined organic phases are washed with sodium chloride solution, dried on sodium sulphate and freed from the solvent. The residue is purified by column chromatography (silica gel, dichloromethane/methanol gradient with 0.1% ammonia). Yield: 4.25 g (37%); mass spectroscopy: [M+H]⁺=365.

c) tert-butyl [3-(4,4-diethyl-2-oxo-4H-benzo[d][1,3]oxazin-1-yl)-1,1-dimethyl-propyl]-carbamate: 2.91 g (9.6 mmol) triphosgene are added at 0 to 5° C. to a solution of 3.50 g (9.6 mmol) tert-butyl {3-[2-(1-ethyl-1-hydroxy-propyl)-phenylamino]-1,1-dimethyl-propyl}-carbamate and 3.37 mL (24 mmol) triethylamine in 35 mL THF. The mixture is left overnight at ambient temperature with stirring and the precipitate formed is suction filtered. The filtrate is evaporated down and the crude product remaining is further reacted directly. Yield: 3.33 g; mass spectroscopy: [M+H]⁺=391.

d) 1-(3-amino-3-methyl-butyl)-4,4-diethyl-1,4-dihydro-benzo[d][1,3]oxazin-2-one: 25 mL trifluoroacetic acid are added dropwise, while being cooled with the ice bath, to a solution of 3.20 g tert-butyl [3-(4,4-diethyl-2-oxo-4H-benzo[d][1,3]oxazin-1-yl)-1,1-dimethyl-propyl]-carbamate (approx. 75%) in 25 mL dichloromethane. The mixture is stirred for 2 hours at ambient temperature, the solvents are distilled off and the acid residues are eliminated by repeated codistillation with toluene. To liberate the free base the residue is combined with 1 molar sodium hydroxide solution and extracted with ethyl acetate. The organic phases are dried on sodium sulphate and evaporated down. The free base is dissolved in 8 mL methanol and combined with ethereal hydrochloric acid. It is stirred overnight and the precipitate formed is suction filtered and washed with diethyl ether. Yield: 2.15 g (hydrochloride); mass spectroscopy: [M+H]⁺=291.

### Intermediate Product 5

### 1-(3-amino-3-methyl-butyl)-spiro(cyclohexane-1,4′-2H-3′,1′-benzoxazin)-2′-one

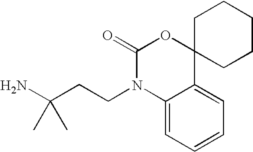

a) 1-(2-nitro-phenyl)-cyclohexanol: 40.16 mL (80.32 mmol) phenylmagnesium chloride (2 M in THF) are added dropwise at −50° C. under nitrogen to a solution of 20.0 g (80.32 mmol) 2-nitro-iodobenzene in 150 mL THF. After 15 minutes stirring 9.98 mL (96.30 mmol) cyclohexanone are quickly added. The reaction mixture is heated to ambient temperature, stirred for two hours and combined with ammonium chloride solution. The aqueous phase is separated off and exhaustively extracted with ethyl acetate. The combined organic phases are washed with sodium chloride solution, dried on sodium sulphate and evaporated down. Column chromatography (silica gel, hexane/ethyl acetate=20:1) yields the product. Yield: 5.20 g (29%); R_{f}=0.26 (silica gel, hexane/ethyl acetate=10:1); ESI-MS: [M+H-H₂O]⁺=204.

b) 1-(2-amino-phenyl)-cyclohexanol: 5.20 g (16.45 mmol) 1-(2-nitro-phenyl)-cyclohexanol in 70 mL ethanol are hydrogenated for 4 hours in the presence of Raney nickel at ambient temperature and 3 bar hydrogen pressure. The catalyst is filtered off through Celite and the filtrate is evaporated down in vacuo. The residue is precipitated from hexane. Yield: 1.53 g (49%); R_{f}=0.38 (silica gel, hexane/ethyl acetate=4:1); ESI-MS: [M+H-H₂O]⁺=174.

c) tert-butyl {3-[2-(1-hydroxy-cyclohexyl)-phenylamino]-1,1-dimethyl-propyl}-carbamate: The compound is obtained analogously to intermediate product 1b from 1-(2-amino-phenyl)-cyclohexanol and tert-butyl (1,1-dimethyl-3-oxo-propyl)-carbamate. Column chromatography (silica gel, hexane/ethyl acetate=7:1) yields the product. Yield: 2.65 g (66%); R_{f}=0.50 (silica gel, hexane/ethyl acetate=4:1).

d) tert-butyl {1,1-dimethyl-3-[spiro(cyclohexane-1,4′-2H-3′,1′-benzoxazin)-2′-oxo-1-yl]-propyl}-carbamate: Prepared analogously to intermediate product 1c from tert-butyl {3-[2-(1-hydroxy-cyclohexyl)-phenylamino]-1,1-dimethyl-propyl}-carbamate. Yield: 2.60 g (92%); R_{f}=0.38 (silica gel, hexane/ethyl acetate 4:1).

e) 1-(3-amino-3-methyl-butyl)-spiro(cyclohexane-1,4′-2H-3′,1′-benzoxazin)-2′-one: Prepared analogously to intermediate product 1d from tert-butyl [1,1-dimethyl-3-(spiro(cyclohexane-1,4′-2H-3′,1′-benzoxazin)-2′-oxo-1-yl)-propyl]-carbamate. Yield: 1.80 g (92%); R_{f}=0.10 (silica gel, dichloromethane/methanol/ammonia=95:5:0.5); ESI-MS: [M+H]⁺=303.

### Intermediate Product 6

### 1-(3-amino-3-methyl-butyl)-4,4-diethyl-8-methoxy-1,4-dihydro-benzo[d][1,3]oxazin-2-one

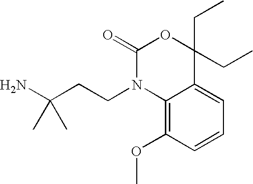

a) 3-(2-amino-3-methoxy-phenyl)-pentan-3-ol: The product is obtained analogously to intermediate product 1a by reacting methyl 2-amino-3-methoxy-benzoate and ethylmagnesium bromide in dichloromethane at −78° C.→RT. Yield: 5.20 g (92%); HPLC-MS: R_{f}=12.85 min. (method A); ESI-MS: [M+H]⁺=210.

b) tert-butyl {3-[2-(1-ethyl-1-hydroxy-propyl)-6-methoxy-phenylamino]-1,1-dimethyl-propyl}-carbamate: The product is obtained analogously to intermediate product 1b starting from 3-(2-amino-3-methoxy-phenyl)-pentan-3-ol and tert-butyl (1,1-dimethyl-3-oxo-propyl)-carbamate. The crude product is purified by column chromatography (silica gel, cyclohexane/ethyl acetate=4:1). Yield: 4.60 g (47%).

c) tert-butyl [3-(4,4-diethyl-8-methoxy-2-oxo-4H-benzo[d][1,3]oxazin-1-yl)-1,1-dimethyl-propyl]-carbamate: The product is obtained analogously to intermediate product 1c starting from tert-butyl {3-[2-(1-ethyl-1-hydroxy-propyl)-6-methoxy-phenylamino]-1,1-dimethyl-propyl}-carbamate. Yield: 4.60 g (94%).

d) 1-(3-amino-3-methyl-butyl)-4,4-diethyl-8-methoxy-1,4-dihydro-benzo[d][1,3]oxazin-2-one: The product is obtained analogously to intermediate product 1d starting from tert-butyl [3-(4,4-diethyl-8-methoxy-2-oxo-4H-benzo[d][1,3]oxazin-1-yl)-1,1-dimethyl-propyl]-carbamate as a free base. Yield: 3.00 g (93%); ESI-MS: [M+H]⁺=321.

### Intermediate Product 7

### 1-(3-amino-3-methyl-butyl)-4,4-diethyl-6-fluoro-1,4-dihydro-benzo[d][1,3]oxazin-2-one

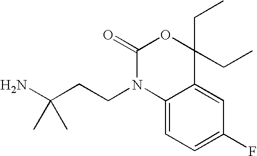

a) 3-(2-amino-5-fluoro-phenyl)-pentan-3-ol: Prepared analogously to intermediate product 1a from methyl 2-amino-5-fluoro-benzoate and ethylmagnesium bromide. The product obtained is purified by chromatography (silica gel, cyclohexane/ethyl acetate=8:1). Yield: 6.00 g (74%).

b) tert-butyl {3-[2-(1-ethyl-1-hydroxy-propyl)-4-fluoro-phenylamino]-,1-dimethyl-propyl}-carbamate: The product is obtained analogously to intermediate product 1b starting from 3-(2-amino-5-fluoro-phenyl)-pentan-3-ol and tert-butyl (1,1-dimethyl-3-oxo-propyl)-carbamate. The crude product is purified by column chromatography (silica gel, hexane/ethyl acetate=6:1 2:1). Yield: 4.50 g (41%).

c) tert-butyl [3-(4,4-diethyl-6-fluoro-2-oxo-4H-benzo[d][1,3]oxazin-1-yl)-1,1-dimethyl-propyl]-carbamate: Prepared analogously to intermediate product 1c from tert-butyl {3-[2-(1-ethyl-1-hydroxy-propyl)-4-fluoro-phenylamino]-1,1-dimethyl-propyl}-carbamate. A difference here is that there is no purification by column chromatography. Yield: 4.8 g.

d) 1-(3-amino-3-methyl-butyl)-4,4-diethyl-6-fluoro-1,4-dihydro-benzo[d][1,3]oxazin-2-one: The target compound is prepared as a free base analogously to intermediate product 1d from tert-butyl [3-(4,4-diethyl-6-fluoro-2-oxo-4H-benzo[d][1,3]oxazin-1-yl)-1,1-dimethyl-propyl]-carbamate. Yield: 3.00 g (99%).

### Example 7

### N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide

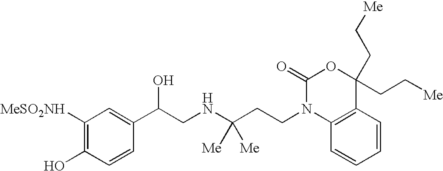

a) N-(2-benzyloxy-5-{2-[1,1-dimethyl-3-(2-oxo-4,4-dipropyl-4H-benzo[d][1,3]oxazin-1-yl)-propylamino]-1-hydroxy-ethyl}-phenyl)-methanesulphonamide: 86 μl (0.619 mmol) triethylamine are added at ambient temperature under a nitrogen atmosphere to a solution of 200 mg (0.564 mmol) 1-(3-amino-3-methyl-butyl)-4,4-dipropyl-1,4-dihydro-benzo[d][1,3]oxazin-2-one hydrochloride in 5 mL THF. The mixture is stirred for 30 minutes, 218 mg (0.575 mmol) N-[2-benzyloxy-5-(2-ethoxy-2-hydroxy-acetyl)-phenyl]-methanesulphonamide are added and the mixture is stirred for a further 2 hours at ambient temperature. The mixture is cooled to 10° C., combined with 51 mg (2.34 mmol) lithium borohydride, heated to ambient temperature and stirred for one hour. It is cooled to 10° C. again and diluted with 15 mL water and 20 mL dichloromethane. The aqueous phase is separated off and extracted with dichloromethane. The combined organic phases are dried on sodium sulphate and evaporated down in vacuo. The residue is dissolved in 8 mL ethyl acetate and acidified to pH 2 by the addition of saturated hydrochloric acid in ethyl acetate. The precipitate formed is filtered off, washed with ethyl acetate and evaporated down. Yield: 260 mg (67%, hydrochloride), HPLC: Rₜ=19.8 minutes (method A).

b) N-(5-{2-[1,1-dimethyl-3-(2-oxo-4,4-dipropyl-4H-benzo[d][1,3]oxazin-1-yl)-propylamino]-1-hydroxy-ethyl}-2-hydroxy-phenyl)-methanesulphonamide: 260 mg (0.386 mmol) N-(2-benzyloxy-5-{2-[1,1-dimethyl-3-(2-oxo-4,4-dipropyl-4H-benzo[d][1,3]oxazin-1-yl)-propylamino]-1-hydroxy-ethyl}-phenyl)-methanesulphonamide hydrochloride in 8 mL methanol are hydrogenated in the presence of 26 mg palladium on charcoal (10%) at ambient temperature. The catalyst is filtered off through Celite and washed with methanol. The filtrate is evaporated down in vacuo and the residue is stirred into diethyl ether. Yield: 120 mg (53%, hydrochloride); mass spectroscopy: [M+H]⁺=548; HPLC: Rₜ=14.7 minutes (method A).

The (R)- and (S)-enantiomers of this embodiment may be obtained by common methods known in the art. The (R)-enantiomer of this embodiment is of particular importance according to the invention.

### Example 8

### N-[5-(2-{1,1-dimethyl-3-[spiro(cyclohexane-1,4′-2H-3′,1′-benzoxazin)-2′-oxo-1-yl]-propylamino}-1-hydroxy-ethyl)-2-hydroxy-phenyl]-methanesulphonamide

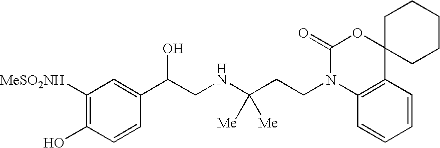

a) N-[2-benzyloxy-5-(2-{3-[spiro(cyclohexane-1,4′-2H-3′,1′-benzoxazin)-2′-oxo-1-yl]-1,1-dimethyl-propylamino}-1-hydroxy-ethyl]-phenyl]-methanesulphonamide: Prepared analogously to the process described for Example 7a from 250 mg (0.66 mmol) N-[2-benzyloxy-5-(2-ethoxy-2-hydroxy-acetyl)-phenyl]-methanesulphonamide and 200 mg (0.66 mmol) 1-(3-amino-3-methyl-butyl)-spiro(cyclohexane-1,4′-2H-3′,1′-benzoxazin)-2′-one. A difference here is that the product obtained as the hydrochloride is also purified by chromatography (silica gel, dichloromethane/methanol=50:1). Yield: 190 mg (46%), HPLC: Rₜ=17.8 minutes (method A).

b) N-[5-(2-{1,1-dimethyl-3-[spiro(cyclohexane-1,4′-2H-3′,1′-benzoxazin)-2′-oxo-1-yl]-propylamino}-1-hydroxy-ethyl)-2-hydroxy-phenyl]-methanesulphonamide: 190 mg (0.31 mmol) N-[2-benzyloxy-5-(2-{3-[spiro(cyclohexane-1,4′-2H-3′,1′-benzoxazin)-2′-oxo-1-yl]-1,1-dimethyl-propylamino}-1-hydroxy-ethyl]-phenyl]-methanesulphonamide are hydrogenated analogously to Example 7b. After separation of the catalyst the filtrate is freed from the solvent, combined with 8 mL ethyl acetate and acidified to pH 2 by the addition of hydrochloric acid in ethyl acetate. The solvent is distilled off and the residue is stirred in diethyl ether and filtered. Yield: 40 mg (23%, hydrochloride); mass spectroscopy: [M+H]⁺=532; HPLC: Rₜ=11.8 minutes (method A).

The (R)- and (S)-enantiomers of this embodiment may be obtained by common methods known in the art. Particular importance attaches to the (R)-enantiomer of this embodiment according to the invention.

### Example 9

### N-[5-(2-{1,1-dimethyl-3-[spiro(cyclopropyl-1,4′-2H-3′,1′-benzoxazin)-2′-oxo-1-yl]-propylamino}-1-hydroxy-ethyl)-2-hydroxy-phenyl]-methanesulphonamide

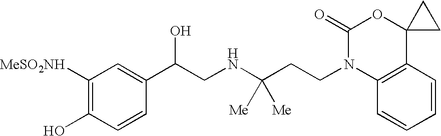

a) N-[2-benzyloxy-5-(2-{3-[spiro(cyclopropyl-1,4′-2H-3′,1′-benzoxazin)-2′-oxo-1-yl]-1,1-dimethyl-propylamino}-1-hydroxy-ethyl]-phenyl]-methanesulphonamide: 292 mg (0.77 mmol) N-[2-benzyloxy-5-(2-ethoxy-2-hydroxy-acetyl)-phenyl]-methanesulphonamide and 200 mg (0.77 mmol) 1-(3-amino-3-methyl-butyl)-spiro(cyclopropyl-1,4′-2H-3′,1′-benzoxazin)-2′-one are reacted and worked up analogously to Example 7a. The crude product is combined with 8 mL ethyl acetate and acidified to pH 2 with hydrochloric acid in ethyl acetate. The solvent is distilled off and the residue is stirred in diethyl ether. Yield: 400 mg (84%, hydrochloride), HPLC: Rₜ=15.2 minutes (method A).

b) N-[5-(2-{1,1-dimethyl-3-[spiro(cyclopropyl-1,4′-2H-3′,1′-benzoxazin)-2′-oxo-1-yl]-propylamino}-1-hydroxy-ethyl)-2-hydroxy-phenyl]-methanesulphonamide: the product is prepared analogously to Example 1b from 400 mg (0.65 mmol) N-[2-benzyloxy-5-(2-{3-[spiro(cyclopropyl-1,4′-2H-3′,1′-benzoxazin)-2′-oxo-1-yl]-1,1-dimethyl-propylamino}-1-hydroxy-ethyl]-phenyl]-methanesulphonamide hydrochloride. Yield: 230 mg (67%, hydrochloride); mass spectroscopy: [M+H]⁺=490; HPLC: Rₜ=8.9 minutes (method A).

The (R)- and (S)-enantiomers of this embodiment may be obtained by common methods known in the art. Particular importance attaches to the (R)-enantiomer of this embodiment according to the invention.

### Example 10

### N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methanesulphonamide:

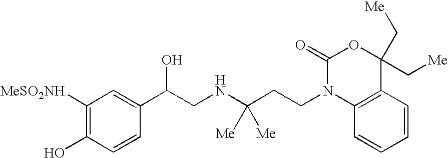

379 mg (1 mmol) N-[2-benzyloxy-5-(2-ethoxy-2-hydroxy-acetyl)-phenyl]-methanesulphonamide and 290 mg (1 mmol) 1-(3-amino-3-methyl-butyl)-4,4-diethyl-1,4-dihydro-benzo[d][1,3]oxazin-2-on are suspended in 5 mL ethanol and heated to 70° C. The resulting solution is stirred for one hour at 70° C. and then cooled to ambient temperature. After the addition of 113 mg (3 mmol) sodium borohydride the mixture is stirred for 3 hours at ambient temperature, combined with 0.7 mL saturated potassium carbonate solution and stirred for a further 30 minutes. The mixture is filtered through aluminium oxide (basic), washed repeatedly with dichloromethane/methanol (15:1) and evaporated down. The crude product thus obtained is purified by chromatography (dichloromethane with 0-10% methanol/ammonia=9:1). The benzylether thus obtained is dissolved in 10 mL methanol and hydrogenated with palladium on charcoal as catalyst at 1 bar hydrogen pressure. Then the catalyst is filtered off and the filtrate is evaporated down. Yield: 338 mg (65% over 2 steps); mass spectroscopy: [M+H]⁺=520.

The (R)- and (S)-enantiomers of this embodiment may be obtained by common methods known in the art. Particular importance attaches to the (R)-enantiomer of this embodiment according to the invention. The angle of rotation of (R)-N-(5-{2-[3-(4,4-diethyl-2-oxo-4H-benzo[d][1,3]oxazin-1-yl)-1,1-dimethyl-propylamino]-1-hydroxy-ethyl}-2-hydroxy-phenyl)-methanesulphonamide hydrochloride (cocrystallised with a molecule of acetone) is −28.8° (c=1%, in methanol at 20° C.).

### Example 11

### N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide

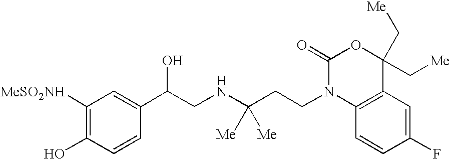

a) N-(2-benzyloxy-5-{2-[3-(4,4-diethyl-6-fluoro-2-oxo-4H-benzo[d][1,3]oxazin-1-yl)-1,1-dimethyl-propylamino]-1-hydroxy-ethyl}-phenyl)-methanesulphonamide: Reaction of 246 mg (0.65 mmol) N-[2-benzyloxy-5-(2-ethoxy-2-hydroxy-acetyl)-phenyl]-methanesulphonamide and 200 mg (0.65 mmol) 1-(3-amino-3-methyl-butyl)-4,4-diethyl-6-fluoro-1,4-dihydro-benzo[D][1,3]oxazin-2-one analogously to Example 7a. One difference is that the preparation of the hydrochloride is omitted. Instead, the free base is purified by chromatography (reverse phase, acetonitrile/water gradient with 0.1% trifluoroacetic acid). Yield: 180 mg (trifluoroacetate), HPLC: R_{f}=17.4 minutes (method A).

b) N-(5-{2-[3-(4,4-diethyl-6-fluoro-2-oxo-4H-benzo[d][1,3]oxazin-1-yl)-1,1-dimethyl-propylamino]-1-hydroxy-ethyl}-2-hydroxy-phenyl)-methanesulphonamide: 175 mg of -N-(2-benzyloxy-5-{2-[3-(4,4-diethyl-6-fluoro-2-oxo-4H-benzo[d][1,3]oxazin-1-yl)-1,1-dimethyl-propylamino]-1-hydroxy-ethyl}-phenyl)-methanesulphonamide trifluoroacetate in 9 mL methanol are hydrogenated in the presence of 40 mg Raney nickel at ambient temperature and 3 bar hydrogen pressure. The catalyst is filtered off and the filtrate is freed from the solvent. Yield: 131 mg (trifluoroacetate); mass spectrometry: [M+H]⁺=538.

The (R)- and (S)-enantiomers of this embodiment may be obtained by common methods known in the art. Particular importance attaches to the (R)-enantiomer of this embodiment according to the invention.

### Example 12

### N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide

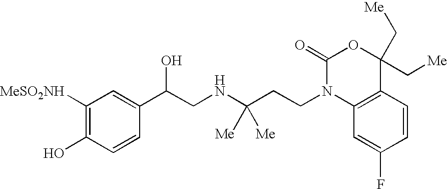

a) N-(2-benzyloxy-5-{2-[3-(4,4-diethyl-7-fluoro-2-oxo-4H-benzo[d][1,3]oxazin-1-yl)-1,1-dimethyl-propylamino]-1-hydroxy-ethyl}-phenyl)-methanesulphonamide: 246 mg (0.65 mmol) N-[2-benzyloxy-5-(2-ethoxy-2-hydroxy-acetyl)-phenyl]-methanesulphonamide and 200 mg (0.65 mmol) 1-(3-amino-3-methyl-butyl)-4,4-diethyl-7-fluoro-1,4-dihydro-benzo[D][1,3]oxazin-2-one were reacted and worked up analogously to Example 7a. A difference is that the production of the hydrochloride is omitted and the free base is purified by chromatography (reverse phase, acetonitrile/water gradient with 0.1% trifluoroacetic acid). Yield: 220 mg (trifluoroacetate), HPLC: Rₜ=17.7 minutes (method A).

b) N-(5-{2-[3-(4,4-diethyl-7-fluoro-2-oxo-4H-benzo[d][1,3]oxazin-1-yl)-1,1-dimethyl-propylamino]-1-hydroxy-ethyl}-2-hydroxy-phenyl)-methanesulphonamide: Prepared analogously to Example 11b from 210 mg of N-(2-benzyloxy-5-{2-[3-(4,4-diethyl-7-fluoro-2-oxo-4H-benzo[d][1,3]oxazin-1-yl)-1,1-dimethyl-propylamino]-1-hydroxy-ethyl}-phenyl)-methanesulphonamide trifluoroacetate. Yield: 154 mg (trifluoroacetate); mass spectrometry: [M+H]⁺=538.

The (R)- and (S)-enantiomers of this embodiment may be obtained by common methods known in the art. Particular importance attaches to the (R)-enantiomer of this embodiment according to the invention.

### Example 13

### N-(5-{2-[3-(4,4-diethyl-8-methoxy-2-oxo-4H-benzo[d][1,3]oxazin-1-yl)-1,1-dimethyl-propylamino]-1-hydroxy-ethyl}-2-hydroxy-phenyl)-methanesulphonamide

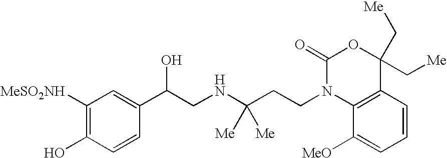

a) N-(2-benzyloxy-5-{2-[3-(4,4-diethyl-8-methoxy-2-oxo-4H-benzo[d][1,3]oxazin-1-yl)-1,1-dimethyl-propylamino]-1-hydroxy-ethyl}-phenyl)-methanesulphonamide: reaction of 237 mg (0.625 mmol) N-[2-benzyloxy-5-(2-ethoxy-2-hydroxy-acetyl)-phenyl]-methanesulphonamide and 200 mg (0.624 mmol) 1-(3-amino-3-methyl-butyl)-4,4-diethyl-8-methoxy-1,4-dihydro-benzo[d][1,3]oxazin-2-one analogously to Example 7a. The crude product is dissolved in ethyl acetate and acidified to pH 2 with hydrochloric acid in ethyl acetate. The solvent is distilled off and the residue is stirred in diethyl ether. Then the hydrochloride thus obtained (330 mg) is further purified by chromatography. Yield: 90 mg (trifluoroacetate), HPLC: Rₜ=17.6 minutes (method A).

b) N-(5-{2-[3-(4,4-diethyl-8-methoxy-2-oxo-4H-benzo[d][1,3]oxazin-1-yl)-1,1-dimethyl-propylamino]-1-hydroxy-ethyl}-2-hydroxy-phenyl)-methanesulphonamide: 80 mg (0.118 mmol) N-(2-benzyloxy-5-{2-[3-(4,4-diethyl-8-methoxy-2-oxo-4H-benzo[d][1,3]oxazin-1-yl)-1,1-dimethyl-propylamino]-1-hydroxy-ethyl}-phenyl)-methanesulphonamide trifluoroacetate are hydrogenated analogously to Example 11b. Yield: 70 mg (trifluoroacetate); mass spectrometry: [M+H]⁺=550.

The (R)- and (S)-enantiomers of this embodiment may be obtained by common methods known in the art. Particular importance attaches to the (R)-enantiomer of this embodiment according to the invention.

### FORMULATION EXAMPLES

### Example 4.1

The following Table shows examples of formulations of the R-enantiomer of the compound of Example 4 according to the invention: 100 ml of pharmaceutical medicament formulation contain, in purified water or water for injections:

| | | benzalkonium | disodium edetate | |
|---|---|---|---|---|
| | 1 (1′-HCl) | chloride | dihydrate | citric acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 100 | 10 | 10 | 3 |
| 9 | 25 | 10 | 10 | 3 |
| 10 | 5 | 10 | 10 | 3 |
| 11 | 0.5 | 15 | 10 | 2 |

### Example 4.2

The following Table shows examples of formulations according to the invention of the R-enantiomer of the compound of Example 4: 100 ml of medicament preparation contain:

| | | | | | | made up to |
|---|---|---|---|---|---|---|
| | | | | | | 100 ml with |
| | | benz- | | disodium | | ethanol/ |
| | 1 | alkonium | | edetate | citric | water |
| | (1′-HCl) | chloride | BHT | dihydrate | acid | mixture |
| Example | (mg) | (mg) | (mg) | (mg) | (mg) | (% V/V) |
| 1 | 10 | 10 | — | 10 | 3 | 20/80 |
| 2 | 10 | 10 | — | 10 | 3 | 50/50 |
| 3 | 1.0 | 5 | — | — | 3 | 70/30 |
| 4 | 100 | — | — | 5 | 5 | 70/30 |
| 5 | 10 | — | — | 5 | 2 | 70/30 |
| 6 | 1.0 | — | 50 | — | 3 | 90/10 |
| 7 | 0.5 | — | — | — | 2 | 90/10 |
| 8 | 1000 | — | — | — | 4 | 90/10 |
| 9 | 100 | — | — | — | 3 | 90/10 |
| 10 | 10 | — | 100 | — | 4 | 95/5 |
| 11 | 2.5 | — | — | — | 3 | 95/5 |
| 12 | 0.5 | — | — | — | 3 | 95/5 |
| 13 | 10 | — | 50 | — | 3 | 100/0 |

### Example 4.3

The following Table shows examples of formulations according to the invention of the R-enantiomer of the compound of Example 4: 100 ml medicament preparation in which the pH has been adjusted to 4 using 1 M HCl contain:

| | | | | | made up to 100 ml |
|---|---|---|---|---|---|
| | | benz- | | disodium | with |
| | 1 | alkonium | | edetate | ethanol/water |
| | (1′-HCl) | chloride | BHT | dihydrate | mixture |
| Example | (mg) | (mg) | (mg) | (mg) | (% V/V) |
| 1 | 10 | 10 | — | 10 | 20/80 |
| 2 | 10 | 10 | — | 10 | 50/50 |
| 3 | 1.0 | 5 | — | — | 70/30 |
| 4 | 100 | — | — | 5 | 70/30 |
| 5 | 10 | — | — | 5 | 70/30 |
| 6 | 1.0 | — | 50 | — | 90/10 |
| 7 | 0.5 | — | — | — | 90/10 |
| 8 | 1000 | — | — | — | 90/10 |
| 9 | 100 | — | — | — | 90/10 |
| 10 | 10 | — | 100 | — | 95/5 |
| 11 | 2.5 | — | — | — | 95/5 |
| 12 | 0.5 | — | — | — | 95/5 |
| 13 | 10 | — | 50 | — | 100/0 |

### Example 5.1

The following Table shows examples of formulations according to the invention of the R-enantiomer of the compound of Example 5: 100 ml medicament formulation contain in purified water or water for injections:

| | | benzalkonium | disodium edetate | |
|---|---|---|---|---|
| | 1 (1′-HCl) | chloride | dihydrate | citric acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 100 | 10 | 10 | 3 |
| 9 | 25 | 10 | 10 | 3 |
| 10 | 5 | 10 | 10 | 3 |
| 11 | 0.5 | 15 | 10 | 2 |

### Example 5.2

The following Table shows examples of formulations according to the invention of the R-enantiomer of the compound of Example 5: 100 ml of medicament preparation contain:

| | | | | | | made up to |
|---|---|---|---|---|---|---|
| | | | | | | 100 ml |
| | | | | | | with |
| | | benz- | | disodium | | ethanol/ |
| | 1 | alkonium | | edetate | citric | water |
| | (1′-HCl) | chloride | BHT | dihydrate | acid | mixture |
| Example | (mg) | (mg) | (mg) | (mg) | (mg) | (% V/V) |
| 1 | 10 | 10 | — | 10 | 3 | 20/80 |
| 2 | 10 | 10 | — | 10 | 3 | 50/50 |
| 3 | 1.0 | 5 | — | — | 3 | 70/30 |
| 4 | 100 | — | — | 5 | 5 | 70/30 |
| 5 | 10 | — | — | 5 | 2 | 70/30 |
| 6 | 1.0 | — | 50 | — | 3 | 90/10 |
| 7 | 0.5 | — | — | — | 2 | 90/10 |
| 8 | 1000 | — | — | — | 4 | 90/10 |
| 9 | 100 | — | — | — | 3 | 90/10 |
| 10 | 10 | — | 100 | — | 4 | 95/5 |
| 11 | 2.5 | — | — | — | 3 | 95/5 |
| 12 | 0.5 | — | — | — | 3 | 95/5 |
| 13 | 10 | — | 50 | — | 3 | 100/0 |

### Example 5.3

The following Table shows examples of formulations according to the invention of the R-enantiomer of the compound of Example 5: 100 ml medicament preparation in which the pH has been adjusted to 4 using 1 M HCl contain:

| | | | | | made up to 100 ml |
|---|---|---|---|---|---|
| | | benz- | | disodium | with |
| | 1 | alkonium | | edetate | ethanol/water |
| | (1′-HCl) | chloride | BHT | dihydrate | mixture |
| Example | (mg) | (mg) | (mg) | (mg) | (% V/V) |
| 1 | 10 | 10 | — | 10 | 20/80 |
| 2 | 10 | 10 | — | 10 | 50/50 |
| 3 | 1.0 | 5 | — | — | 70/30 |
| 4 | 100 | — | — | 5 | 70/30 |
| 5 | 10 | — | — | 5 | 70/30 |
| 6 | 1.0 | — | 50 | — | 90/10 |
| 7 | 0.5 | — | — | — | 90/10 |
| 8 | 1000 | — | — | — | 90/10 |
| 9 | 100 | — | — | — | 90/10 |
| 10 | 10 | — | 100 | — | 95/5 |
| 11 | 2.5 | — | — | — | 95/5 |
| 12 | 0.5 | — | — | — | 95/5 |
| 13 | 10 | — | 50 | — | 100/0 |

### Example 6.1

The following Table shows examples of formulations according to the invention of the R-enantiomer of the compound of Example 6: 100 ml medicament formulation contain in purified water or water for injections:

| | | benzalkonium | disodium edetate | |
|---|---|---|---|---|
| | 1 (1′-HCl) | chloride | dihydrate | citric acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 100 | 10 | 10 | 3 |
| 9 | 25 | 10 | 10 | 3 |
| 10 | 5 | 10 | 10 | 3 |
| 11 | 0.5 | 15 | 10 | 2 |

### Example 6.2

The following Table shows examples of formulations according to the invention of the R-enantiomer of the compound of Example 6: 100 ml of medicament preparation contain:

| | | | | | | made up to |
|---|---|---|---|---|---|---|
| | | | | | | 100 ml with |
| | | benz- | | disodium | | ethanol/ |
| | 1 | alkonium | | edetate | citric | water |
| | (1′-HCl) | chloride | BHT | dihydrate | acid | mixture |
| Example | (mg) | (mg) | (mg) | (mg) | (mg) | (% V/V) |
| 1 | 10 | 10 | — | 10 | 3 | 20/80 |
| 2 | 10 | 10 | — | 10 | 3 | 50/50 |
| 3 | 1.0 | 5 | — | — | 3 | 70/30 |
| 4 | 100 | — | — | 5 | 5 | 70/30 |
| 5 | 10 | — | — | 5 | 2 | 70/30 |
| 6 | 1.0 | — | 50 | — | 3 | 90/10 |
| 7 | 0.5 | — | — | — | 2 | 90/10 |
| 8 | 1000 | — | — | — | 4 | 90/10 |
| 9 | 100 | — | — | — | 3 | 90/10 |
| 10 | 10 | — | 100 | — | 4 | 95/5 |
| 11 | 2.5 | — | — | — | 3 | 95/5 |
| 12 | 0.5 | — | — | — | 3 | 95/5 |
| 13 | 10 | — | 50 | — | 3 | 100/0 |

### Example 6.3

The following Table shows examples of formulations according to the invention of the R-enantiomer of the compound of Example 6: 100 ml medicament preparation in which the pH has been adjusted to 4 using 1 M HCl contain:

| | | | | | made up to 100 ml |
|---|---|---|---|---|---|
| | | benz- | | disodium | with |
| | 1 | alkonium | | edetate | ethanol/water |
| | (1′-HCl) | chloride | BHT | dihydrate | mixture |
| Example | (mg) | (mg) | (mg) | (mg) | (% V/V) |
| 1 | 10 | 10 | — | 10 | 20/80 |
| 2 | 10 | 10 | — | 10 | 50/50 |
| 3 | 1.0 | 5 | — | — | 70/30 |
| 4 | 100 | — | — | 5 | 70/30 |
| 5 | 10 | — | — | 5 | 70/30 |
| 6 | 1.0 | — | 50 | — | 90/10 |
| 7 | 0.5 | — | — | — | 90/10 |
| 8 | 1000 | — | — | — | 90/10 |
| 9 | 100 | — | — | — | 90/10 |
| 10 | 10 | — | 100 | — | 95/5 |
| 11 | 2.5 | — | — | — | 95/5 |
| 12 | 0.5 | — | — | — | 95/5 |
| 13 | 10 | — | 50 | — | 100/0 |

### Example 7.1

The following Table shows examples of formulations according to the invention of the R-enantiomer of the compound of Example 7: 100 ml medicament formulation contain in purified water or water for injections:

| | | benzalkonium | disodium edetate | |
|---|---|---|---|---|
| | 1 (1′-HCl) | chloride | dihydrate | citric acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 100 | 10 | 10 | 3 |
| 9 | 25 | 10 | 10 | 3 |
| 10 | 5 | 10 | 10 | 3 |
| 11 | 0.5 | 15 | 10 | 2 |

### Example 7.2

The following Table shows examples of formulations according to the invention of the R-enantiomer of the compound of Example 7: 100 ml of medicament preparation contain:

| | | | | | | made up to |
|---|---|---|---|---|---|---|
| | | | | | | 100 ml with |
| | | benz- | | disodium | | ethanol/ |
| | 1 | alkonium | | edetate | citric | water |
| | (1′-HCl) | chloride | BHT | dihydrate | acid | mixture |
| Example | (mg) | (mg) | (mg) | (mg) | (mg) | (% V/V) |
| 1 | 10 | 10 | — | 10 | 3 | 20/80 |
| 2 | 10 | 10 | — | 10 | 3 | 50/50 |
| 3 | 1.0 | 5 | — | — | 3 | 70/30 |
| 4 | 100 | — | — | 5 | 5 | 70/30 |
| 5 | 10 | — | — | 5 | 2 | 70/30 |
| 6 | 1.0 | — | 50 | — | 3 | 90/10 |
| 7 | 0.5 | — | — | — | 2 | 90/10 |
| 8 | 1000 | — | — | — | 4 | 90/10 |
| 9 | 100 | — | — | — | 3 | 90/10 |
| 10 | 10 | — | 100 | — | 4 | 95/5 |
| 11 | 2.5 | — | — | — | 3 | 95/5 |
| 12 | 0.5 | — | — | — | 3 | 95/5 |
| 13 | 10 | — | 50 | — | 3 | 100/0 |

### Example 7.3

The following Table shows examples of formulations according to the invention of the R-enantiomer of the compound of Example 7: 100 ml medicament preparation in which the pH has been adjusted to 4 using 1 M HCl contain:

| | | | | | made up to 100 ml |
|---|---|---|---|---|---|
| | | benz- | | disodium | with |
| | 1 | alkonium | | edetate | ethanol/water |
| | (1′-HCl) | chloride | BHT | dihydrate | mixture |
| Example | (mg) | (mg) | (mg) | (mg) | (% V/V) |
| 1 | 10 | 10 | — | 10 | 20/80 |
| 2 | 10 | 10 | — | 10 | 50/50 |
| 3 | 1.0 | 5 | — | — | 70/30 |
| 4 | 100 | — | — | 5 | 70/30 |
| 5 | 10 | — | — | 5 | 70/30 |
| 6 | 1.0 | — | 50 | — | 90/10 |
| 7 | 0.5 | — | — | — | 90/10 |
| 8 | 1000 | — | — | — | 90/10 |
| 9 | 100 | — | — | — | 90/10 |
| 10 | 10 | — | 100 | — | 95/5 |
| 11 | 2.5 | — | — | — | 95/5 |
| 12 | 0.5 | — | — | — | 95/5 |
| 13 | 10 | — | 50 | — | 100/0 |

### Example 8.1

The following Table shows examples of formulations according to the invention of The R-enantiomer of the compound of Example 8: 100 ml medicament formulation contain, in purified water or water for injections:

| | | benzalkonium | disodium edetate | |
|---|---|---|---|---|
| | 1 (1′-HCl) | chloride | dihydrate | citric acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 100 | 10 | 10 | 3 |
| 9 | 25 | 10 | 10 | 3 |
| 10 | 5 | 10 | 10 | 3 |
| 11 | 0.5 | 15 | 10 | 2 |

### Example 8.2

The following Table shows examples of formulations according to the invention of the R-enantiomer of the compound of Example 8: 100 ml of medicament preparation contain:

| | | | | | | made up to |
|---|---|---|---|---|---|---|
| | | | | | | 100 ml with |
| | | benz- | | disodium | | ethanol/ |
| | 1 | alkonium | | edetate | citric | water |
| | (1′-HCl) | chloride | BHT | dihydrate | acid | mixture |
| Example | (mg) | (mg) | (mg) | (mg) | (mg) | (% V/V) |
| 1 | 10 | 10 | — | 10 | 3 | 20/80 |
| 2 | 10 | 10 | — | 10 | 3 | 50/50 |
| 3 | 1.0 | 5 | — | — | 3 | 70/30 |
| 4 | 100 | — | — | 5 | 5 | 70/30 |
| 5 | 10 | — | — | 5 | 2 | 70/30 |
| 6 | 1.0 | — | 50 | — | 3 | 90/10 |
| 7 | 0.5 | — | — | — | 2 | 90/10 |
| 8 | 1000 | — | — | — | 4 | 90/10 |
| 9 | 100 | — | — | — | 3 | 90/10 |
| 10 | 10 | — | 100 | — | 4 | 95/5 |
| 11 | 2.5 | — | — | — | 3 | 95/5 |
| 12 | 0.5 | — | — | — | 3 | 95/5 |
| 13 | 10 | — | 50 | — | 3 | 100/0 |

### Example 8.3

The following Table shows examples of formulations according to the invention of the R-enantiomer of the compound of Example 8: 100 ml medicament preparation in which the pH has been adjusted to 4 using 1 M HCl contain:

| | | | | | made up to 100 ml |
|---|---|---|---|---|---|
| | | benz- | | disodium | with |
| | 1 | alkonium | | edetate | ethanol/water |
| | (1′-HCl) | chloride | BHT | dihydrate | mixture |
| Example | (mg) | (mg) | (mg) | (mg) | (% V/V) |
| 1 | 10 | 10 | — | 10 | 20/80 |
| 2 | 10 | 10 | — | 10 | 50/50 |
| 3 | 1.0 | 5 | — | — | 70/30 |
| 4 | 100 | — | — | 5 | 70/30 |
| 5 | 10 | — | — | 5 | 70/30 |
| 6 | 1.0 | — | 50 | — | 90/10 |
| 7 | 0.5 | — | — | — | 90/10 |
| 8 | 1000 | — | — | — | 90/10 |
| 9 | 100 | — | — | — | 90/10 |
| 10 | 10 | — | 100 | — | 95/5 |
| 11 | 2.5 | — | — | — | 95/5 |
| 12 | 0.5 | — | — | — | 95/5 |
| 13 | 10 | — | 50 | — | 100/0 |

### Example 9.1

The following Table shows examples of formulations according to the invention of the R-enantiomer of the compound of Example 9: 100 ml medicament formulation contain in purified water or water for injections:

| | | benzalkonium | disodium edetate | |
|---|---|---|---|---|
| | 1 (1′-HCl) | chloride | dihydrate | citric acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 100 | 10 | 10 | 3 |
| 9 | 25 | 10 | 10 | 3 |
| 10 | 5 | 10 | 10 | 3 |
| 11 | 0.5 | 15 | 10 | 2 |

### Example 9.2

The following Table shows examples of formulations according to the invention of the R-enantiomer of the compound of Example 9: 100 ml of medicament preparation contain:

| | | | | | | made up |
|---|---|---|---|---|---|---|
| | | | | | | to 100 ml |
| | | | | | | with |
| | | | | disodium | | ethanol/ |
| | 1 | benzalkonium | | edetate | citric | water |
| Exam- | (1′-HCl) | chloride | BHT | dihydrate | acid | mixture |
| ple | (mg) | (mg) | (mg) | (mg) | (mg) | (% V/V) |
| 1 | 10 | 10 | — | 10 | 3 | 20/80 |
| 2 | 10 | 10 | — | 10 | 3 | 50/50 |
| 3 | 1.0 | 5 | — | — | 3 | 70/30 |
| 4 | 100 | — | — | 5 | 5 | 70/30 |
| 5 | 10 | — | — | 5 | 2 | 70/30 |
| 6 | 1.0 | — | 50 | — | 3 | 90/10 |
| 7 | 0.5 | — | — | — | 2 | 90/10 |
| 8 | 1000 | — | — | — | 4 | 90/10 |
| 9 | 100 | — | — | — | 3 | 90/10 |
| 10 | 10 | — | 100 | — | 4 | 95/5 |
| 11 | 2.5 | — | — | — | 3 | 95/5 |
| 12 | 0.5 | — | — | — | 3 | 95/5 |
| 13 | 10 | — | 50 | — | 3 | 100/0 |

### Example 9.3

The following Table shows examples of formulations according to the invention of the R-enantiomer of the compound of Example 9: 100 ml medicament preparation in which the pH has been adjusted to 4 using 1 M HCl contain:

| | | | | | made up to |
|---|---|---|---|---|---|
| | | | | | 100 ml |
| | | | | disodium | with |
| | 1 | benzalkonium | | edetate | ethanol/water |
| | (1′-HCl) | chloride | BHT | dihydrate | mixture |
| Example | (mg) | (mg) | (mg) | (mg) | (% V/V) |
| 1 | 10 | 10 | — | 10 | 20/80 |
| 2 | 10 | 10 | — | 10 | 50/50 |
| 3 | 1.0 | 5 | — | — | 70/30 |
| 4 | 100 | — | — | 5 | 70/30 |
| 5 | 10 | — | — | 5 | 70/30 |
| 6 | 1.0 | — | 50 | — | 90/10 |
| 7 | 0.5 | — | — | — | 90/10 |
| 8 | 1000 | — | — | — | 90/10 |
| 9 | 100 | — | — | — | 90/10 |
| 10 | 10 | — | 100 | — | 95/5 |
| 11 | 2.5 | — | — | — | 95/5 |
| 12 | 0.5 | — | — | — | 95/5 |
| 13 | 10 | — | 50 | — | 100/0 |

### Example 10.1

The following Table shows examples of formulations according to the invention of the R-enantiomer of the compound of Example 10: 100 ml medicament formulation contain, in purified water or water for injections:

| | | benzalkonium | disodium edetate | |
|---|---|---|---|---|
| | 1 (1′-HCl) | chloride | dihydrate | citric acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 100 | 10 | 10 | 3 |
| 9 | 25 | 10 | 10 | 3 |
| 10 | 5 | 10 | 10 | 3 |
| 11 | 0.5 | 15 | 10 | 2 |

### Example 10.2

The following Table shows examples of formulations according to the invention of the R-enantiomer of the compound of Example 10: 100 ml of medicament preparation contain:

| | | | | | | made up |
|---|---|---|---|---|---|---|
| | | | | | | to 100 ml |
| | | | | | | with |
| | | | | disodium | | ethanol/ |
| | 1 | benzalkonium | | edetate | citric | water |
| Exam- | (1′-HCl) | chloride | BHT | dihydrate | acid | mixture |
| ple | (mg) | (mg) | (mg) | (mg) | (mg) | (% V/V) |
| 1 | 10 | 10 | — | 10 | 3 | 20/80 |
| 2 | 10 | 10 | — | 10 | 3 | 50/50 |
| 3 | 1.0 | 5 | — | — | 3 | 70/30 |
| 4 | 100 | — | — | 5 | 5 | 70/30 |
| 5 | 10 | — | — | 5 | 2 | 70/30 |
| 6 | 1.0 | — | 50 | — | 3 | 90/10 |
| 7 | 0.5 | — | — | — | 2 | 90/10 |
| 8 | 1000 | — | — | — | 4 | 90/10 |
| 9 | 100 | — | — | — | 3 | 90/10 |
| 10 | 10 | — | 100 | — | 4 | 95/5 |
| 11 | 2.5 | — | — | — | 3 | 95/5 |
| 12 | 0.5 | — | — | — | 3 | 95/5 |
| 13 | 10 | — | 50 | — | 3 | 100/0 |

### Example 10.3

The following Table shows examples of formulations according to the invention of the R-enantiomer of the compound of Example 10: 100 ml medicament preparation in which the pH has been adjusted to 4 using 1 M HCl contain:

| | | | | | made up to |
|---|---|---|---|---|---|
| | | | | | 100 ml |
| | | | | disodium | with |
| | 1 | benzalkonium | | edetate | ethanol/water |
| | (1′-HCl) | chloride | BHT | dihydrate | mixture |
| Example | (mg) | (mg) | (mg) | (mg) | (% V/V) |
| 1 | 10 | 10 | — | 10 | 20/80 |
| 2 | 10 | 10 | — | 10 | 50/50 |
| 3 | 1.0 | 5 | — | — | 70/30 |
| 4 | 100 | — | — | 5 | 70/30 |
| 5 | 10 | — | — | 5 | 70/30 |
| 6 | 1.0 | — | 50 | — | 90/10 |
| 7 | 0.5 | — | — | — | 90/10 |
| 8 | 1000 | — | — | — | 90/10 |
| 9 | 100 | — | — | — | 90/10 |
| 10 | 10 | — | 100 | — | 95/5 |
| 11 | 2.5 | — | — | — | 95/5 |
| 12 | 0.5 | — | — | — | 95/5 |
| 13 | 10 | — | 50 | — | 100/0 |

### Example 11.1

The following Table shows examples of formulations according to the invention of the R-enantiomer of the compound of Example 11: 100 ml medicament formulation contain, in purified water or water for injections:

| | | benzalkonium | disodium edetate | |
|---|---|---|---|---|
| | 1 (1′-HCl) | chloride | dihydrate | citric acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 100 | 10 | 10 | 3 |
| 9 | 25 | 10 | 10 | 3 |
| 10 | 5 | 10 | 10 | 3 |
| 11 | 0.5 | 15 | 10 | 2 |

### Example 11.2

The following Table shows examples of formulations according to the invention of the R-enantiomer of the compound of Example 11: 100 ml of medicament preparation contain:

| | | | | | | made up |
|---|---|---|---|---|---|---|
| | | | | | | to 100 ml |
| | | | | | | with |
| | | | | disodium | | ethanol/ |
| | 1 | benzalkonium | | edetate | citric | water |
| Exam- | (1′-HCl) | chloride | BHT | dihydrate | acid | mixture |
| ple | (mg) | (mg) | (mg) | (mg) | (mg) | (% V/V) |
| 1 | 10 | 10 | — | 10 | 3 | 20/80 |
| 2 | 10 | 10 | — | 10 | 3 | 50/50 |
| 3 | 1.0 | 5 | — | — | 3 | 70/30 |
| 4 | 100 | — | — | 5 | 5 | 70/30 |
| 5 | 10 | — | — | 5 | 2 | 70/30 |
| 6 | 1.0 | — | 50 | — | 3 | 90/10 |
| 7 | 0.5 | — | — | — | 2 | 90/10 |
| 8 | 1000 | — | — | — | 4 | 90/10 |
| 9 | 100 | — | — | — | 3 | 90/10 |
| 10 | 10 | — | 100 | — | 4 | 95/5 |
| 11 | 2.5 | — | — | — | 3 | 95/5 |
| 12 | 0.5 | — | — | — | 3 | 95/5 |
| 13 | 10 | — | 50 | — | 3 | 100/0 |

### Example 11.3

The following Table shows examples of formulations according to the invention of the R-enantiomer of the compound of Example 11: 100 ml medicament preparation in which the pH has been adjusted to 4 using 1 M HCl contain:

| | | | | | made up to |
|---|---|---|---|---|---|
| | | | | | 100 ml |
| | | | | disodium | with |
| | 1 | benzalkonium | | edetate | ethanol/water |
| | (1′-HCl) | chloride | BHT | dihydrate | mixture |
| Example | (mg) | (mg) | (mg) | (mg) | (% V/V) |
| 1 | 10 | 10 | — | 10 | 20/80 |
| 2 | 10 | 10 | — | 10 | 50/50 |
| 3 | 1.0 | 5 | — | — | 70/30 |
| 4 | 100 | — | — | 5 | 70/30 |
| 5 | 10 | — | — | 5 | 70/30 |
| 6 | 1.0 | — | 50 | — | 90/10 |
| 7 | 0.5 | — | — | — | 90/10 |
| 8 | 1000 | — | — | — | 90/10 |
| 9 | 100 | — | — | — | 90/10 |
| 10 | 10 | — | 100 | — | 95/5 |
| 11 | 2.5 | — | — | — | 95/5 |
| 12 | 0.5 | — | — | — | 95/5 |
| 13 | 10 | — | 50 | — | 100/0 |

### Example 12.1

The following Table shows examples of formulations according to the invention of the R-enantiomer of the compound of Example 12: 100 ml medicament formulation contain in purified water or water for injections:

| | | benzalkonium | disodium edetate | |
|---|---|---|---|---|
| | 1 (1′-HCl) | chloride | dihydrate | citric acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 100 | 10 | 10 | 3 |
| 9 | 25 | 10 | 10 | 3 |
| 10 | 5 | 10 | 10 | 3 |
| 11 | 0.5 | 15 | 10 | 2 |

### Example 12.2

The following Table shows examples of formulations according to the invention of the R-enantiomer of the compound of Example 12: 100 ml of medicament preparation contain:

| | | | | | | made up |
|---|---|---|---|---|---|---|
| | | | | | | to 100 ml |
| | | | | | | with |
| | | | | disodium | | ethanol/ |
| | 1 | benzalkonium | | edetate | citric | water |
| Exam- | (1′-HCl) | chloride | BHT | dihydrate | acid | mixture |
| ple | (mg) | (mg) | (mg) | (mg) | (mg) | (% V/V) |
| 1 | 10 | 10 | — | 10 | 3 | 20/80 |
| 2 | 10 | 10 | — | 10 | 3 | 50/50 |
| 3 | 1.0 | 5 | — | — | 3 | 70/30 |
| 4 | 100 | — | — | 5 | 5 | 70/30 |
| 5 | 10 | — | — | 5 | 2 | 70/30 |
| 6 | 1.0 | — | 50 | — | 3 | 90/10 |
| 7 | 0.5 | — | — | — | 2 | 90/10 |
| 8 | 1000 | — | — | — | 4 | 90/10 |
| 9 | 100 | — | — | — | 3 | 90/10 |
| 10 | 10 | — | 100 | — | 4 | 95/5 |
| 11 | 2.5 | — | — | — | 3 | 95/5 |
| 12 | 0.5 | — | — | — | 3 | 95/5 |
| 13 | 10 | — | 50 | — | 3 | 100/0 |

### Example 12.3

The following Table shows examples of formulations according to the invention of the R-enantiomer of the compound of Example 12: 100 ml medicament preparation in which the pH has been adjusted to 4 using 1 M HCl contain:

| | | | | | made up to |
|---|---|---|---|---|---|
| | | | | | 100 ml |
| | | | | disodium | with |
| | 1 | benzalkonium | | edetate | ethanol/water |
| | (1′-HCl) | chloride | BHT | dihydrate | mixture |
| Example | (mg) | (mg) | (mg) | (mg) | (% V/V) |
| 1 | 10 | 10 | — | 10 | 20/80 |
| 2 | 10 | 10 | — | 10 | 50/50 |
| 3 | 1.0 | 5 | — | — | 70/30 |
| 4 | 100 | — | — | 5 | 70/30 |
| 5 | 10 | — | — | 5 | 70/30 |
| 6 | 1.0 | — | 50 | — | 90/10 |
| 7 | 0.5 | — | — | — | 90/10 |
| 8 | 1000 | — | — | — | 90/10 |
| 9 | 100 | — | — | — | 90/10 |
| 10 | 10 | — | 100 | — | 95/5 |
| 11 | 2.5 | — | — | — | 95/5 |
| 12 | 0.5 | — | — | — | 95/5 |
| 13 | 10 | — | 50 | — | 100/0 |

### Example 13.1

The following Table shows examples of formulations according to the invention of the R-enantiomer of the compound of Example 13: 100 ml medicament formulation contain, in purified water or water for injections:

| | | benzalkonium | disodium edetate | |
|---|---|---|---|---|
| | 1 (1′-HCl) | chloride | dihydrate | citric acid |
| Example | (mg) | (mg) | (mg) | (mg) |
| 1 | 10 | 10 | — | 3 |
| 2 | 1.0 | 15 | — | 5 |
| 3 | 100 | — | — | 5 |
| 4 | 10 | — | 5 | 3 |
| 5 | 1.0 | — | 10 | 3 |
| 6 | 0.5 | 5 | 7 | 2 |
| 7 | 1000 | 5 | 15 | 4 |
| 8 | 100 | 10 | 10 | 3 |
| 9 | 25 | 10 | 10 | 3 |
| 10 | 5 | 10 | 10 | 3 |
| 11 | 0.5 | 15 | 10 | 2 |

### Example 13.2

The following Table shows examples of formulations according to the invention of the R-enantiomer of the compound of Example 13: 100 ml of medicament preparation contain:

| | | | | | | made up |
|---|---|---|---|---|---|---|
| | | | | | | to 100 ml |
| | | | | | | with |
| | | | | disodium | | ethanol/ |
| | 1 | benzalkonium | | edetate | citric | water |
| Exam- | (1′-HCl) | chloride | BHT | dihydrate | acid | mixture |
| ple | (mg) | (mg) | (mg) | (mg) | (mg) | (% V/V) |
| 1 | 10 | 10 | — | 10 | 3 | 20/80 |
| 2 | 10 | 10 | — | 10 | 3 | 50/50 |
| 3 | 1.0 | 5 | — | — | 3 | 70/30 |
| 4 | 100 | — | — | 5 | 5 | 70/30 |
| 5 | 10 | — | — | 5 | 2 | 70/30 |
| 6 | 1.0 | — | 50 | — | 3 | 90/10 |
| 7 | 0.5 | — | — | — | 2 | 90/10 |
| 8 | 1000 | — | — | — | 4 | 90/10 |
| 9 | 100 | — | — | — | 3 | 90/10 |
| 10 | 10 | — | 100 | — | 4 | 95/5 |
| 11 | 2.5 | — | — | — | 3 | 95/5 |
| 12 | 0.5 | — | — | — | 3 | 95/5 |
| 13 | 10 | — | 50 | — | 3 | 100/0 |

### Example 13.3

The following Table shows examples of formulations according to the invention of the R-enantiomer of the compound of Example 13: 100 ml medicament preparation in which the pH has been adjusted to 4 using 1 M HCl contain:

| | | | | | made up to |
|---|---|---|---|---|---|
| | | | | | 100 ml |
| | | | | disodium | with |
| | 1 | benzalkonium | | edetate | ethanol/water |
| | (1′-HCl) | chloride | BHT | dihydrate | mixture |
| Example | (mg) | (mg) | (mg) | (mg) | (% V/V) |
| 1 | 10 | 10 | — | 10 | 20/80 |
| 2 | 10 | 10 | — | 10 | 50/50 |
| 3 | 1.0 | 5 | — | — | 70/30 |
| 4 | 100 | — | — | 5 | 70/30 |
| 5 | 10 | — | — | 5 | 70/30 |
| 6 | 1.0 | — | 50 | — | 90/10 |
| 7 | 0.5 | — | — | — | 90/10 |
| 8 | 1000 | — | — | — | 90/10 |
| 9 | 100 | — | — | — | 90/10 |
| 10 | 10 | — | 100 | — | 95/5 |
| 11 | 2.5 | — | — | — | 95/5 |
| 12 | 0.5 | — | — | — | 95/5 |
| 13 | 10 | — | 50 | — | 100/0 |

1) Medicament formulation comprising as the sole active substance one or more compounds of general formula 1 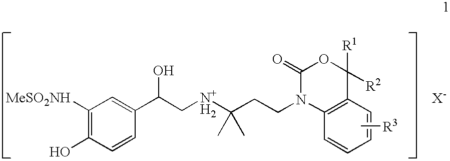
wherein
R¹ and R² which may be identical or different, denote hydrogen, halogen, C₁₋₄-alkyl or together denote -C₁₋₆-alkylene and
R³ denotes hydrogen, halogen, OH, C₁₋₄-alkyl or O-C₁₋-alkyl;
X⁻ denotes a mono- or polysubstituted negatively charged anion, and at least one pharmacologically acceptable acid.

2) The medicament formulation according to claim 1, further comprising pharmacologically acceptable excipients.

3) The medicament formulation according to claim 1, further comprising complexing agent.

4) The medicament formulation according to claim 1, further comprising water, ethanol or a mixture of water and ethanol as solvent.

5) The medicament formulation according to claim 1, wherein said active substance is in the form of the tautomers, enantiomers, mixtures of enantiomers, racemates or solvates thereof.

6) The medicament formulation according to claim 1, wherein
R¹ and R² which may be identical or different, denote hydrogen, fluorine, chlorine, methyl, ethyl, propyl, butyl or together denote —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, chlorine, OH, methyl, ethyl, methoxy, or ethoxy;
X⁻ is a mono- or polysubstituted negatively charged anion.

7) The medicament formulation according to claim 1, wherein said active substance is in the form of the tautomers, enantiomers, mixtures of enantiomers, racemates or solvates thereof.

8) The medicament formulation according to claim 1, wherein
R¹ and R² which may be identical or different, denote hydrogen, methyl, ethyl, propyl or together denote —CH₂—CH₂, —CH₂—CH₂—CH₂, —CH₂—CH₂—CH₂—CH₂ or —CH₂—CH₂—CH₂—CH₂—CH₂—;
R³ denotes hydrogen, fluorine, OH, methyl or methoxy;
X⁻ is a mono- or polysubstituted negatively charged anion selected from chloride, bromide, sulphate, methanesulphonate, maleate, acetate, benzoate, citrate, salicylate, trifluoroacetate, fumarate, tartrate and succinate.

9) The medicament formulation according to claim 8, wherein said active substance is in the form of the tautomers, enantiomers, mixtures of enantiomers, racemates or solvates thereof.

10) The medicament formulation according to claim 1, wherein the pharmacologically acceptable acid is selected from the inorganic acids hydrochloric acid, hydrobromic acid, nitric acid, sulphuric acid and phosphoric acid or from the organic acids ascorbic acid, citric acid, malic acid, tartaric acid, maleic acid, succinic acid, fumaric acid, acetic acid, formic acid, propionic acid, methanesulphonic acid and benzenesulphonic acid.

11) The medicament formulation according to claims 1, wherein said formulation has a pH of 2.5 to 6.5.

12) The medicament formulation according to claim 1, wherein said formulation further comprises benzalkonium chloride as excipient.

13) The medicament formulation according to claim 12, wherein the content of benzalkonium chloride is 1 to 50 mg per 100 ml solution.

14) The medicament formulation according to claim 1, wherein the content of 1′ is about 0.1 to 1600 mg per 100 ml solution.

15) The medicament formulation according to claim 1, further comprising a complexing agent as a further ingredient.

16) The medicament formulation according to claim 15, wherein the content of complexing agent is 1 to 50 mg per 100 ml solution.

17) The medicament formulation according to claim 1, wherein said formulation comprises pure water as solvent.

18) The medicament formulation according to claim 1, wherein said formulation comprises pure ethanol as solvent.

19) The medicament formulation according to claim 1, wherein said formulation comprises a mixture of water and ethanol as solvent.

20) The medicament formulation according to claim 19, wherein the percentage amount of ethanol by mass is in the range between 5 and 99% ethanol.

21) The medicament formulation according to claim 1, wherein said formulation comprises an antioxidant as a further ingredient.

22) The medicament formulation according to claim 1, wherein said antioxidant is ascorbic acid, propylgallate, BHA (butylhydroxyanisol), BHT (butylhydroxytoluene), TBHQ (tert-butylhydroxyquinone), vitamin A, vitamin E or α-tocopherol.

23) Medicament formulation comprising as the sole active substance a free base of formula 1′ 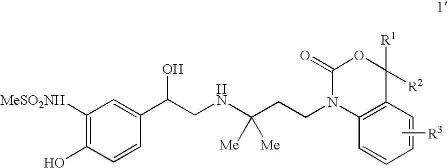
wherein the groups R¹, R² and R³ may have the meanings given in claim 1; and at least one pharmacologically acceptable acid.

24) The medicament formulation according to claim 23, wherein said formulation further comprises pharmacologically acceptable excipients.

25) The medicament formulation according to claim 23, wherein said formulation further comprises complexing agent.

26) The medicament formulation according to claim 23, wherein said formulation further comprises water, ethanol or a mixture of water and ethanol as solvent.

27) The medicament formulation according to claim 23, wherein said active substance is in the form of the tautomers, enantiomers, mixtures of enantiomers, racemates or solvates thereof.

28) A method of treating respiratory complaints comprising administering to a patient in need thereof a therapeutically effective amount of a medicament formulation according to claim 1.

29) Inhalation kit consisting of a medicament formulation according to claim 1 and an inhaler suitable for nebulising this medicament formulation.

30) Inhalation kit according to claim 29, wherein the inhaler is a Respimat®.
